SEQUENCE LISTING (9 sequences)

SEQ ID NO: 1 - protein, 202 aa, Listeria monocytogenes
MGYIGTKRSERSQDAIEDYEVPLNHFNKDLIQAFIDENEAYDTLKTKKVRLWKFVAPRAGATSWHHTGTYYNKTDHYSLEKVADELLQNGDEWEEQFKAYVKEEQETATSEPVFLSVIKVQIWGGSMKRPKLVGHEVVMGVKKEGWLHAVSKATQSKYKLSANKVEMQKHYSLEDYSALTKDFPEFKAQKRAINKKMKEMYN

SEQ ID NO: 2 - protein, 204 aa, Enterococcus faecalis
MGYVGKSRSVRSQIAIDNAEVPLNHITKDYILTFVTENNIDETLKNESVAMWKFVAKRHGSTSWHHVSKHYNKIDHYDLHDVAEYFSMNYDSLKNDYQNLLDQKRQAKNDLIKNLKLGIIKVQIWGGTKRYPKLEGYESVMGVVKDGWLHTVTLSNQTKYKITGNKIEEITIFELDQYDILTKKFPEFRAMKRKINKEVARLSK

SEQ ID NO: 3 - protein, 109 aa, Enterococcus faecalis
MAILNNKGEKISIDCADLISEVEEDILIFGGTFLVYAICSWREIEQVEYISDYVHADNPESYKDELTTKEYAELKEIYEKDLEELKITKNKQMNLNELLSILTIQNSIT

SEQ ID NO: 4 - protein, 100 aa, Streptococcus gallolyticus
MKISVDSEKLLNEAINDFDIFGEDFNVYAIYSYREDYDFEYISDYVDADEPTRDEFETEEDYQEVMKDFKENLDSLKFTKHKKMTIADLVHELWEQNRIF

SEQ ID NO: 5 - protein, 182 aa, Streptococcus macedonicus
MKIDTTVTEVKENGKTYLRLLKGNEQLKAVSDKAVAGVNLFPGAKIGSFLVRQDNIVVFPDNKGEFDLDFFNLLNDNFETLVEYAKMADCLDIAFDINEKSYFNMIMWLMKNIDENWSQSPYGESFYSSKDIDWGYKPEGSLRVSDHWNFGQDGEHCPTAEPVDGWAVCKFENGKYHLIKKF

SEQ ID NO: 6 - protein, 181 aa, Streptococcus gallolyticus
MKIDTTVTEVKENGKTYLRLVEGTEQLKAISDKAMAGVNLFPGAKIDSFLVKQDSIVVFPDNKGEFDLDFFKQLDENFDTIAKYARVATCFEEVAFDEKSYFNMIMWLMDNMDENWSQSPYGESFYSSKNIDWGYKPEGSLRVSDHWNFGENGEHCPTAEPVDGWAVCKFENGKYHLIKKF

SEQ ID NO: 7 - protein, 124 aa, Staphylococcus simulans
MKLIVEVEETNYKNLVNYTKLTNESHNILVNRLISEYITKPYELRLDLSERYSNRDLIEFKFMLIEYCKEALQDIKELANSDEAYETDEAFEAVFRQLFEEVISNPDTVLKAFHSYTSFLEENK

SEQ ID NO: 8 - protein, 115 aa, Staphylococcus pseudintermedius
MKLIINIEDKNYKYLTELAQQDNTNIGSIVNNLIQTHITDVNESYRSVDKKELDEFSRVMQHYFHEDLASMYDVIGSDEELSTDKQMLKVYKKLYQDVALRNGIALELFNAYKKG

SEQ ID NO: 9 - protein, 6 aa, Artificial Sequence
  Description of Artificial Sequence Synthetic 6xHis tag
HHHHHH

(19) United States
(12) Patent Application Publication
(10) Pub. No.: US 2022/0380421 A1
(43) Pub. Date: Dec. 1, 2022

(54) BROAD SPECTRUM INHIBITORS OF CRISPR-CAS9

(71) Applicant: The Regents of the University of California, Oakland, CA (US)

(72) Inventors: Joseph Bondy-Denomy, Oakland, CA (US); Caroline Mahendra, Oakland, CA (US)

(21) Appl. No.: 17/734,775

(22) Filed: May 2, 2022

Related U.S. Application Data
(63) Continuation of application No. PCT/US2020/059531, filed on Nov. 6, 2020.
(60) Provisional application No. 62/932,383, filed on Nov. 7, 2019.

(51) Int. Cl. C07K 14/47; C07K 14/195; C12N 15/85; C12N 9/22

(52) U.S. Cl. CPC C07K 14/4703; C07K 14/195; C12N 15/85; C12N 9/22

(57) ABSTRACT

The present disclosure provides Cas9-inhibiting polypeptides and polynucleotides, and methods of using the same to inhibit Cas9 in cells.

### CROSS REFERENCE TO RELATED APPLICATIONS

The present application is a Continuation of International Patent Application No. PCT/US2020/059531, filed Nov. 6, 2020, which claims priority to U.S. Provisional Patent Application No. 62/932,383, filed on Nov. 7, 2019, each of which is incorporated herein by reference in its entirety.

### STATEMENT AS TO RIGHTS TO INVENTIONS MADE UNDER FEDERALLY SPONSORED RESEARCH AND DEVELOPMENT

This invention was made with government support under grants OD021344 and R01 GM127489 awarded by the National Institutes of Health, and grant HR0011-17-2-0043 awarded by the Defense Advanced Research Projects Agency. The government has certain rights in the invention.

### SEQUENCE LISTING

The instant application contains a Sequence Listing which has been submitted electronically in ASCII format and is hereby incorporated by reference in its entirety. Said ASCII copy, created on Jul. 13, 2022, is named 081906-1325406-236710US_SL.txt and is 11,528 bytes in size.

### BACKGROUND

Bacteria are constantly exposed to invasive mobile genetic elements (MGEs) that can either benefit or harm the host. Many MGEs encode antibiotic resistance pathogenicity factors that can enhance microbe virulence (Palmer et al., 2010; Waldor and Mekalanos, 1996), although most are regarded as parasitic entities (Koonin, 2016). To combat MGE invasions, bacteria possess defense mechanisms, including restriction modification and CRISPR-Cas adaptive immunity (Labrie et al., 2010), which can limit the exchange of destructive genetic material (Price et al., 2016; Edgar and Qimron, 2010; Zhang et al., 2013). CRISPR-Cas systems are widespread, found in roughly half of bacteria and over 80% of archaea (Makarova et al., 2015), and can protect host genomes against phage infection and plasmid conjugation (Garneau et al., 2010). Nevertheless, the occurrence of horizontal gene transfer (HGT) persists across species, as is evident by DNA sequence estimates suggesting that 5-6% of genes in bacterial genomes are derived from HGT (Clark and Pazdernik, 2013).

Bacteriophages have responded to CRISPR-Cas with anti-CRISPR (Acr) proteins (Bondy-Denomy et al., 2013), which can inhibit CRISPR-Cas complex formation/stability (Harrington et al., 2019; Zhu et al., 2019), target DNA binding, or cleavage (Bondy-Denomy et al., 2015; Dong et al., 2019; Knott et al., 2019). To date, 46 distinct families against various CRISPR-Cas subtypes have been discovered, of which type II-A Cas9 inhibitors alone constitute 11 (Rauch et al., 2017; Hynes et al., 2017, 2018; Uribe et al., 2019; Forsberg et al., 2019). Numerous strategies have been employed for Acr discovery, including bioinformatic (Pawluk et al., 2016; Rauch et al., 2017), experimental (Bondy-Denomy et al.; 2013, Hynes et al., 2017), and metagenomic screening (Uribe et al., 2019; Forsberg et al., 2019). Many of these approaches have discovered Acrs on phages and prophages. It is not clear, however, how other MGEs avoid CRISPR targeting. In the opportunistic pathogen Enterococcus faecalis, for example, where integrated conjugative elements (ICEs) encode antibiotic resistance, their presence is associated with non-functional CRISPR-Cas systems (Palmer and Gilmore, 2010; Hullahalli et al., 2018). It is unclear whether Acrs play a role in the horizontal spread and vertical maintenance of non-phage MGEs by compromising the host immune defense systems.

The present disclosure provides previously unknown CRISPR-Cas9 inhibitors from plasmids and other conjugative elements in Firmicutes bacteria. The present inhibitors are encoded by mobile genetic elements in bacteria and possess a wide range of inhibition capacity, making them suitable for use as broad regulators of different Cas9 nucleases.

### BRIEF SUMMARY OF THE INVENTION

In one aspect, the present disclosure provides a method of inhibiting a Cas9 polypeptide in a cell, the method comprising, introducing a Cas9-inhibiting polypeptide into a cell, wherein: the Cas9-inhibiting polypeptide is heterologous to the cell, and the Cas9-inhibiting polypeptide is substantially (e.g., at least about 60%, 70%, 80%, 90%, 95%, 96%, 97%, 98%, 99%) identical to any one or more of SEQ ID NOS: 1-8; thereby inhibiting the Cas9 polypeptide in a cell.

In some embodiments, the method comprises contacting the Cas9-inhibiting polypeptide with a Cas9 polypeptide in the cell. In some embodiments, the Cas9-inhibiting polypeptide comprises one of SEQ ID NOS: 1-8. In some embodiments, the Cas9-inhibiting polypeptide comprises SEQ ID NO: 1, 2, 4 or 7. In some embodiments, the cell comprises an expression cassette comprising a promoter operably linked to a polynucleotide encoding the Cas9 polypeptide. In some embodiments, the cell comprises the Cas9 polypeptide before the introducing. In some such embodiments, the promoter is inducible and the method comprises contacting the cell with an agent or condition that induces expression of the Cas9 polypeptide in the cell prior to the introducing of the Cas9-inhibiting polypeptide. In some embodiments, the cell comprises the Cas9 polypeptide after the introducing of the Cas9-inhibiting polypeptide. In some such embodiments, the promoter is inducible and the method comprises contacting the cell with an agent or condition that induces expression of the Cas9 polypeptide in the cell after the introducing of the Cas9-inhibiting polypeptide.

In some embodiments of the method, the introducing of the Cas9-inhibiting polypeptide comprises expressing the Cas9-inhibiting polypeptide in the cell from an expression cassette that is present in the cell and is heterologous to the cell, wherein the expression cassette comprises a promoter operably linked to a polynucleotide encoding the Cas9-inhibiting polypeptide. In some embodiments, the promoter is an inducible promoter and the introducing of the Cas9-inhibiting polypeptide comprises contacting the cell with an agent that induces expression of the Cas9-inhibiting polypeptide. In some embodiments, the introducing of the Cas9-inhibiting polypeptide comprises introducing an RNA encoding the Cas9-inhibiting polypeptide into the cell and expressing the Cas9-inhibiting polypeptide in the cell from the RNA. In some embodiments, the introducing of the Cas9-inhibiting polypeptide comprises inserting the Cas9-inhibiting polypeptide into the cell or contacting the cell with the Cas9-inhibiting polypeptide.

In some embodiments of the method, the cell is a eukaryotic cell. In some embodiments, the cell is a mammalian cell. In some embodiments, the cell is a human cell. In some embodiments, the cell is a blood cell or an induced pluripotent stem cell. In some embodiments, the method occurs ex vivo. In some such embodiments, the cells are introduced into a mammal after the introducing of the Cas9-inhibiting polypeptide, and optionally after the contacting of the Cas9 polypeptide. In some embodiments, the cells are autologous to the mammal.

In some embodiments of the method, the cell is a prokaryotic cell. In some such embodiments, the introducing comprises introducing a polynucleotide encoding the Cas9-inhibiting polypeptide into the cell using bacteriophage, and expressing the Cas9-inhibiting polypeptide in the cell from the polynucleotide. In some embodiments of any of the herein-described methods, the Cas9 polypeptide is SpyCas9, Efa1Cas9, or Efa3Cas9.

In another aspect, the present disclosure provides a cell comprising a Cas9-inhibiting polypeptide, wherein the Cas9-inhibiting polypeptide is heterologous to the cell and the Cas9-inhibiting polypeptide is substantially (e.g., at least about 60%, 70%, 80%, 90%, 95%, 96%, 97%, 98%, 99%) identical to any one or more of SEQ ID NOS: 1-8.

In some embodiments, the cell is a eukaryotic cell. In some embodiments, the cell is a mammalian cell. In some embodiments, the cell is a human cell. In some embodiments, the cell is a prokaryotic cell.

In another aspect, the present disclosure provides a polynucleotide comprising a nucleic acid encoding a Cas9-inhibiting polypeptide, wherein the Cas9-inhibiting polypeptide is substantially (e.g., at least about 60%, 70%, 80%, 90%, 95%, 96%, 97%, 98%, 99%) identical to any one or more of SEQ ID NOS: 1-8.

In some embodiments, the Cas9-inhibiting polypeptide inhibits one or more Cas9 polypeptides selected from the group consisting of SpyCas9, Efa1Cas9, and Efa3Cas9. In some embodiments, the polynucleotide is RNA. In some embodiments, the polynucleotide is DNA.

In another aspect, the present disclosure provides an expression cassette comprising any of the herein-described polynucleotides encoding a Cas9-inhibiting polypeptide, operably linked to a promoter. In some embodiments, the promoter is heterologous to the polynucleotide encoding the Cas9-inhibiting polypeptide. In some embodiments, the promoter is inducible.

In another aspect, the present disclosure provides a vector comprising any of the herein-described expression cassettes. In some embodiments, the vector is a viral vector.

In another aspect, the present disclosure provides a bacteriophage comprising any of the herein-described expression cassettes.

In another aspect, the present disclosure provides an isolated Cas9-inhibiting polypeptide, wherein the Cas9-inhibiting polypeptide is substantially (e.g., at least about 60%, 70%, 80%, 90%, 95%, 96%, 97%, 98%, 99%) identical to any one or more of SEQ ID NOS:1-8. In some embodiments, the Cas9-inhibiting polypeptide inhibits one or more Cas9 polypeptides selected from the group consisting of SpyCas9, Efa1Cas9, and Efa3Cas9.

In another aspect, the present disclosure provides a pharmaceutical composition comprising any of the herein-described Cas9-inhibiting polypeptides or polynucleotides encoding a Cas9-inhibiting polypeptide.

In another aspect, the present disclosure provides a delivery vehicle comprising any of the herein-described Cas9-inhibiting polypeptides or polynucleotides encoding a Cas9-inhibiting polypeptide. In some embodiments, the delivery vehicle is a liposome or nanoparticle.

### BRIEF DESCRIPTION OF THE DRAWINGS

FIGS. 1A-1C. Identification of four new Type II-A inhibitors, AcrIIA16-19. FIG. 1A: Schematic representation of Type II-A acr genes, with vertical arrows indicating relationships between acr loci and percent protein sequence identity. Numbers in genes correspond to AcrIIA number. Grey genes are proteins of unknown function that tested negative for AcrIIA activity. FIG. 1B: Schematic of phage plaque assays to assess CRISPR-SpyCas9 inhibition. 10-fold serial dilutions of targeted phage (black circles) are spotted on a lawn of P. aeruginosa (grey background) expressing the Type II-A CRISPR-Cas system and indicated acr genes. CRISPR strength is determined by expression of sgRNA from the chromosome (low), or from a multicopy plasmid at increasing induction levels [0.1, 1, 10 mM IPTG]. ΔCRISPR lacks a phage-targeting sgRNA. EV, empty vector. FIG. 1C: Schematic of CRISPRi to assess AcrIIA inhibition of dCas9 binding to target DNA. Chromosomally-integrated dCas9 (yellow asterisks) in P. aeruginosa programmed to bind the phzM gene promoter with sgRNA expressed from a multicopy plasmid at low or medium IPTG induction levels, in the presence of indicated AcrIIA proteins. CRISPRi inhibition was assessed by quantification of pyocyanin levels in response to dCas9 phzM gene repression, relative to ΔCRISPR. Representative pictures of at least three biological replicates at medium CRISPR strength are shown (bottom).

FIGS. 2A-2C. Prevalence of acrIIA genes in integrative mobile genetic elements and their effect on CRISPR-targeting during conjugation. FIG. 2A: Left: Host distribution of acrIIA16-19 based on phylogenetic analysis, see FIG. 5A. Right: Mobile genetic element distribution of acrIIA16-19 based on genomic neighbors characteristic of phage or plasmid genes. Unknown/Host denote genomic regions that could not be identified as either phage or plasmid-like elements. FIG. 2B: Schematic of conjugation in E. faecalis encoding a Type II-A CRISPR system that targets the protospacer-bearing plasmid in the presence of indicated acrIIA genes episomally expressed in recipient cells. Conjugation frequency is quantified as transconjugants per donor relative to a non-targeted plasmid. FIG. 2C: Schematic of plasmid conjugation in E. faecalis from a donor to recipient. The conjugating plasmid carries the indicated acrIIA gene and is targeted by the host's Type II-A CRISPR-Cas system.

FIGS. 3A-3D. In vitro binding and inhibition activities of AcrIIA16-19 against SpyCas9. FIG. 3A: Time courses of SpyCas9 cleavage reactions targeting a double-stranded linear DNA template in the presence of purified Acr proteins. (L) 1 kb dsDNA ladder, (—) DNA template alone. FIG. 3B: Immunoprecipitation (IP) of Myc-tagged SpyCas9-sgRNA. Left: Immunoblot probed with α-Myc (top), α-GST (middle), and α-E. coli RNA polymerase β as a loading control (bottom). Image is cropped to show only the bands corresponding to full-length SpyCas9, see FIG. 7B for uncropped version. Right: SDS-PAGE analysis and Coomassie staining. FIG. 3C: Time courses of target DNA cleavage reactions using SpyCas9 co-immunoprecipitated with AcrIIA-proteins from FIG. 3B. Top band present in EV, AcrIIA14, 15 and 16 lanes are co-purifying nucleic acid contaminants. (L) 1 kb dsDNA ladder, (−) DNA template alone. FIG. 3D: Immunoprecipitation (IP) of GST-Acr proteins in the presence of Myc-tagged SpyCas9 either sgRNA-bound (left) or Apo- without sgRNA (right). Immunoblot for Myc-Cas9 (top) or GST-Acr (bottom).

FIGS. 4A-4B. Schematic of acr loci and lethal self-genome cleavage assay. FIG. 4A: Full schematic of acr loci with relevant neighboring genes displayed. FIG. 4B: Schematic of SpyCas9 in P. aeruginosa programmed to cause lethal self-genome cleavage to assess bacterial survival in the presence of AcrIIA proteins. CRISPR strength is determined by titrating levels of IPTG, which induces expression of sgRNA targeting the chromosomal phzM gene from a multicopy plasmid.

FIGS. 5A-5D. Anti-CRISPR distribution in integrative mobile genetic elements across bacterial taxa. Phylogenetic analysis of acrIIA16-19 homologs (FIG. 5A to 5D, respectively) reconstructed from a midpoint rooted minimum-evolution of full-length protein sequences identified following an iterative PSI-BLASTp search. Branches are labeled with species name and colored according to species class (see legend). Species for which AcrIIA homologs have been tested in this study are shown in bold.

FIGS. 6A-6D. AcrIIA enhance conjugation-mediated horizontal gene transfer in E. faecalis; related to FIG. 2. FIG. 6A: Schematic of the native CRISPR-Cas system in E. faecalis strains OG1RF for CRISPR1 and T11RF for CRISPR3 utilized for all conjugation experiments. Black diamonds denote spacers in the CRISPR array and red indicates spacer that match the protospacer in the targeted plasmids. FIGS. 6B, 6C: Mating outcomes during plasmid conjugation of a targeted plasmid from donor to recipient cells where indicated acrIIA genes are (FIG. 6B) pre-expressed in recipient cells, or (FIG. 6C) encoded on conjugating plasmid. Data displayed as 10-fold colony serial dilution spots of donor, recipient or transconjugant cells on selective antibiotic plates. FIG. 6D: Schematic of E. faecalis conjugation of protospacer and acrIIA-bearing plasmid transferring into CRISPR-defective recipients. For CRISPR1, the bona fide AcrIIA4 is utilized to suppress CRISPR-targeting, and a ΔCas9 strain from previously reported work is used for CRISPR3 (Price et al., 2016). Red * denotes plasmids that have lost conjugation ability.

FIGS. 7A-7C. AcrIIA16-19 biochemical analysis, related to FIG. 3. FIG. 7A: Coomassie-stained polyacrylamide gel showing AcrIIA proteins purified from E. coli. AcrIIA proteins are eluted from Heparin or Ni-NTA columns as indicated and fractionated by SEC. FIG. 7B: Uncropped version of FIG. 3B, displaying all fragments of SpyCas9 present and both Myc and GST pulldowns. FIG. 7C: Immunoblot of Myc and GST pulldowns from P. aeruginosa expressing GST-tagged AcrIIA proteins and Myc-tagged Apo-SpyCas9.

### DETAILED DESCRIPTION

### 1. Introduction

The present disclosure provides new polypeptide inhibitors of Cas9 nuclease (“Cas9-inhibiting polypeptides”), and methods of using the Cas9-inhibiting polypeptides, that have been identified from plasmids and other conjugative elements in Firmicutes bacteria. These Cas9-inhibiting polypeptides are designated AcrIIA16, AcrIIA17, AcrIIA18, and AcrIIA19. AcrIIA16 corresponds, e.g., to SEQ ID NOS: 1 and 2 (showing AcrIIA16 from Listeria monocytogenes and Enterococcus faecalis, respectively); AcrIIA17 corresponds to, e.g., SEQ ID NOS: 3 and 4 (showing AcrIIA17 from Enterococcus faecalis and Streptococcus gallolyticus, respectively); AcrIIA18 corresponds to, e.g., SEQ ID NOS: 5 and 6 (showing AcrIIA18 from Streptococcus macedonicus and Streptococcus gallolyticus, respectively); and AcrIIA19 corresponds to, e.g., SEQ ID NO: 7 and 8 (showing AcrIIA19 from Staphylococcus simulans and Staphylococcus pseudintermedius, respectively).

The Cas9-inhibiting polypeptides described herein possess a wide range of inhibition capacity, inhibiting, for example, one or more of SpyCas9 (i.e., Cas9 from Streptococcus pyogenes), CRISPR1 from Enterococcus (Efa1Cas9), and CRISPR3 from Enterococcus (Efa3Cas9), and as such can be used to regulate multiple different Cas9 proteins, including those often used for gene editing. For example, the proteins can be used as broad-spectrum inhibitors, providing a single option for providing a Cas9 “off-switch” in vivo.

The present polypeptides can be used in numerous ways to inhibit unwanted Cas9 activity. For example, the proteins can be used to limit excess Cas9 nuclease activity and thereby enhance the specificity of Cas9. They can be used to protect organisms against Cas9-mediated genome manipulations in the wild, such as gene drives. The proteins can also be used to reduce virulence of infectious pathogens that possess functional CRISPR-Cas9 systems. The proteins are also useful for engineering into phage therapeutics to enhance their potency. These and other uses and features of the proteins are described in more detail elsewhere herein.

### 2. Definitions

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the protein” includes reference to one or more proteins known to those skilled in the art, and so forth.

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.8X, 0.81X, 0.82X, 0.83X, 0.84X, 0.85X, 0.86X, 0.87X, 0.88X, 0.89X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, 1.1X, 1.11X, 1.12X, 1.13X, 1.14X, 1.15X, 1.16X, 1.17X, 1.18X, 1.19X, and 1.2X. Thus, “about X” is intended to teach and provide written description support for a claim limitation of, e.g., “0.98X.”

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

“AcrIIA16” refers to a Cas9 inhibitor protein, e.g., a protein comprising the amino acid sequence shown as SEQ ID NO:1 or SEQ ID NO:2, or a protein comprising an amino acid sequence substantially identical to SEQ ID NO:1 or SEQ ID NO:2, e.g., a protein comprising 70%, 75%, 80%, 85%, 90%, 95%, 96%, 97%, 98%, 99% or more identity to SEQ ID NO: 1 or SEQ ID NO:2, or variants, derivatives, or fragments of any of these proteins. AcrIIA16 proteins can be from any source, and can bind to and/or inhibit Cas9 proteins, including, e.g., SpyCas9, Efa1Cas9, Efa3Cas9, and others, e.g., as assessed in vitro, in prokaryotic or eukaryotic cells, or in vivo. AcrIIA16 can refer to an AcrIIA16 protein from any organism, e.g., Listeria monocytogenes (IIA16-Lmo, e.g., SEQ ID NO: 1 or Accession no. WP_061665674.1) or Enterococcus faecalis (IIA16-Efa; e.g., SEQ ID NO: 2 or Accession no. WP_025188019.1).

“AcrIIA17” refers to a Cas9 inhibitor protein, e.g., a protein comprising the amino acid sequence shown as SEQ ID NO:3 or SEQ ID NO:4, or a protein comprising an amino acid sequence substantially identical to SEQ ID NO:3 or SEQ ID NO:4, e.g., a protein comprising 70%, 75%, 80%, 85%, 90%, 95%, 96%, 97%, 98%, 99% or more identity to SEQ ID NO: 3 or SEQ ID NO:4, or variants, derivatives, or fragments of any of these proteins. AcrIIA17 proteins can bind to and/or inhibit Cas9 proteins, including, e.g., SpyCas9, Efa1Cas9, Efa3Cas9, and others, e.g., as assessed in vitro, in prokaryotic or eukaryotic cells, or in vivo. AcrIIA17 can refer to an AcrIIA17 from any organism, e.g., Enterococcus faecalis (IIA17-Efa; e.g., SEQ ID NO: 3 or Accession no. WP_002401839.1) or Streptococcus gallolyticus (IIA17-Sga; e.g., SEQ ID NO: 4 or Accession no. WP_074626943.1).

AcrIIA18 refers to a Cas9 inhibitor protein, e.g., a protein comprising the amino acid sequence shown as SEQ ID NO:5 or SEQ ID NO:6, or a protein comprising an amino acid sequence substantially identical to SEQ ID NO:5 or SEQ ID NO:6, e.g., a protein comprising 70%, 75%, 80%, 85%, 90%, 95%, 96%, 97%, 98%, 99% or more identity to SEQ ID NO:5 or SEQ ID NO:6, or variants, derivatives, or fragments of any of these proteins. AcrIIA18 proteins can be from any source, and can bind to and/or inhibit Cas9 proteins, including, e.g., SpyCas9, Efa1Cas9, Efa3Cas9, and others, e.g., as assessed in vitro, in prokaryotic or eukaryotic cells, or in vivo. AcrIIA18 can refer to an AcrIIA18 from any organism, e.g. Streptococcus macedonicus (IIA18-Sma; e.g., SEQ ID NO: 5 or Accession no. WP_099390844.1) or Streptococcus gallolyticus (IIA18-Sga; e.g., SEQ ID NO: 6 or Accession no. WP_074627086.1).

AcrIIA19 refers to a Cas9 inhibitor protein, e.g., a protein comprising the amino acid sequence shown as SEQ ID NO:7 or SEQ ID NO:8, or a protein comprising an amino acid sequence substantially identical to SEQ ID NO:7 or SEQ ID NO:8, e.g., a protein comprising 70%, 75%, 80%, 85%, 90%, 95%, 96%, 97%, 98%, 99% or more identity to SEQ ID NO:7 or SEQ ID NO:8, or variants, derivatives, or fragments of any of these proteins. AcrIIA19 proteins can be from any source, and can bind to and inhibit Cas9 proteins, including, e.g., SpyCas9, Efa1Cas9, Efa3Cas9, and others, e.g., as assessed in vitro, in prokaryotic or eukaryotic cells, or in vivo. AcrIIA19 can refer to an AcrIIA19 from any organism, e.g. Staphylococcus simulans (IIA19-Ssim; e.g., SEQ ID NO: 7 or Accession no. WP_107591702.1) or Staphylococcus pseudintermedius (IIA19-Spse; e.g., SEQ ID NO: 8 or Accession no. WP_100006909.1).

The term “gene” means the segment of DNA involved in producing a polypeptide chain. It may include regions preceding and following the coding region (leader and trailer) as well as intervening sequences (introns) between individual coding segments (exons).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. The promoter can be a heterologous promoter.

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter. The promoter can be a heterologous promoter. In the context of promoters operably linked to a polynucleotide, a “heterologous promoter” refers to a promoter that would not be so operably linked to the same polynucleotide as found in a product of nature (e.g., in a wild-type organism).

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. In some cases, conservatively modified variants of Cas9 or sgRNA can have an increased stability, assembly, or activity as described herein.

The following eight groups each contain amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

### 8) Cysteine (C), Methionine (M)

(see, e.g., Creighton, Proteins, W. H. Freeman and Co., N. Y. (1984)).

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

In the present application, amino acid residues are numbered according to their relative positions from the left-most residue, which is numbered 1 in an unmodified wild-type polypeptide sequence.

As used in herein, the terms “identical” or percent “identity,” in the context of describing two or more polynucleotide or amino acid sequences, refer to two or more sequences or specified subsequences that are the same. Two sequences that are “substantially identical” have at least 60% identity, preferably 65%, 70%, 75%, 80%, 85%, 90%, 91%, 92%, 93, 94%, 95%, 96%, 97%, 98%, 99%, or 100% identity, when compared and aligned for maximum correspondence over a comparison window, or designated region as measured using a sequence comparison algorithm or by manual alignment and visual inspection where a specific region is not designated. With regard to polynucleotide sequences, this definition also refers to the complement of a test sequence. With regard to amino acid sequences, in some cases, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST 2.0 algorithm and the default parameters discussed below are used.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned.

An algorithm for determining percent sequence identity and sequence similarity is the BLAST 2.0 algorithm, which are described in Altschul et al., (1990) J. Mol. Biol. 215: 403-410. Software for performing BLAST analyses is publicly available at the National Center for Biotechnology Information website, ncbi.nlm.nih.gov. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits acts as seeds for initiating searches to fmd longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The “CRISPR-Cas” system refers to a class of bacterial systems for defense against foreign nucleic acid. CRISPR-Cas systems are found in a wide range of eubacterial and archaeal organisms. CRISPR-Cas systems include type I, II, III, V, and VI sub-types. Wild-type type II CRISPR-Cas systems utilize the RNA-mediated nuclease, Cas9 in complex with guide and activating RNA to recognize and cleave foreign nucleic acid.

Cas9 homologs are found in a wide variety of eubacteria, including, but not limited to bacteria of the following taxonomic groups: Actinobacteria, Aquificae, Bacteroidetes-Chlorobi, Chlamydiae-Verrucomicrobia, Chlroflexi, Cyanobacteria, Firmicutes, Proteobacteria, Spirochaetes, and Thermotogae. An exemplary Cas9 polypeptide is the Streptococcus pyogenes Cas9 polypeptide (SpyCas9). Additional Cas9 proteins and homologs thereof are described in, e.g., Chylinksi, et al., RNA Biol. 2013 May 1; 10(5): 726-737; Nat. Rev. Microbiol. 2011 June; 9(6): 467-477; Hou, et al., Proc Natl Acad Sci USA (2013) Sep. 24; 110(39):15644-9; Sampson et al., Nature. 2013 May 9; 497(7448):254-7; and Jinek, et al., Science. 2012 Aug. 17; 337(6096):816-21. The Cas9 protein can be nuclease defective. For example, the Cas9 protein can be a nicking endonuclease that nicks target DNA, but does not cause double strand breakage. Cas9 can also have both nuclease domains deactivated to generate “dead Cas9” (dCas9), a programmable DNA-binding protein with no nuclease activity. In some embodiments, dCas9 DNA-binding is inhibited by the polypeptides described herein.

### 3. Cas9 Inhibitors

As set forth in the present disclosure, including the examples and sequence listing, a number of Cas9-inhibiting polypeptides have been discovered and are provided herein. Examples of exemplary Cas9-inhibiting polypeptides include proteins comprising an amino acid sequence selected from any of SEQ ID NOs: 1-8 or a fragment thereof, or an amino acid sequence substantially (e.g., at least about 50, 60, 70, 75, 80, 85, 90, 95, 96, 97, 98, or 99%) identical to any of SEQ ID NOS: 1-8 or a fragment thereof. In some embodiments, the polypeptides, in addition to having one of the above-listed sequences, will include other amino acid sequences or other chemical moieties (e.g., detectable labels) at the amino terminus, carboxyl terminus, or both. Additional amino acid sequences can include, but are not limited to tags, detectable markers, or nuclear localization signal sequences. In some embodiments, the Cas9-inhibiting polypeptides inhibit one or more Cas9 polypeptides selected from the group consisting of SpyCas9, Efa1Cas9, and Efa3Cas9. In some embodiments, the Cas9-inhibiting polypeptide is an AcrIIA16 polypeptide. In some embodiments, the Cas9-inhibiting polypeptide is an AcrIIA17 polypeptide. In some embodiments, the Cas9-inhibiting polypeptide is an AcrIIA18 polypeptide. In some embodiments, the Cas9-inhibiting polypeptide is an AcrIIA19 polypeptide.

As used herein, a “Cas9-inhibiting polypeptide” refers to a protein that can inhibit the binding or activity of a Cas9 protein (including dCas9) through any mechanism, e.g., by inhibiting the formation or stability of a CRISPR-Cas complex (i.e., Cas9 with a guide RNA), by inhibiting its binding to a target DNA, or by inhibiting cleavage of the target DNA. A Cas9-inhibiting polypeptide could inhibit any of these activities by, e.g., 10%, 25%, 50%, 75%, 90%, or more. The function of the Cas9 protein can be assessed in one or more assays or systems, including in vitro (e.g., inhibiting Cas9 nuclease or DNA-binding activity) or in cells. For example, a Cas9 inhibiting polypeptide can be used to inhibit a heterologous Cas9, e.g., SpyCas9 in Pseudomonas aeruginosa, against bacteriophage challenge or in a self-targeting tolerance assay. They can also be used to inhibit Cas9 activity in a natural host such as Enterococcus. They can also be used to reduce gene editing by various Cas9 orthologs in human cell lines.

In some embodiments, the Cas9 inhibiting activity of an inhibitor is assayed in a bacteriophage plaque assay. When cells expressing Cas9 and a guide RNA are infected by bacteriophages bearing a targeted DNA sequence and protospacer adjacent motif (PAM), the infection event is prevented by Cas9, limiting the emergence of bacteriophage replicative plaques. This is compared to a bacteriophage lacking the targeted DNA sequence and to a bacteriophage infecting a strain expressing a non-targeting guide RNA, which produces normal sized colonies when used to transform the same strain. The expression of a Cas9 inhibitor, however, neutralizes Cas9 activity and leads to bacteriophage plaques. While it is believed the Cas9-inhibiting polypeptides' inhibitory activity can be measured in other ways, the above assay, presented in more detail in the Examples, is the assay for determining whether the Cas9-inhibiting polypeptide has activity.

Table 1A presents the amino acid sequences and accession numbers of the present Cas9-inhibiting polypeptides, and, as shown in Table 1B, the present Cas9-inhibiting polypeptides show a broad spectrum of activity and can inhibit a range of Cas9 proteins, including SpyCas9 (from Streptococcus pyogenes) and EfaCas9 from Enterococcus, both the CRISPR1 (SpyCas9-like) and the CRISPR3 (SauCas9-like) systems. These Cas9 families include the main families being used in human gene editing therapeutic applications. It is believed and expected that the Cas9-inhibiting polypeptides described herein will also similarly inhibit other Cas9 proteins. As such, due to their broad specificity, a single or reduced number of the present broad spectrum inhibitors could be used as a single option for gene editing “off switches” in vivo. Such an ability provides a significant improvement over current known inhibitors of Cas9, which are restricted to specific subtypes and would thus need to be used in combination in order to provide broad Cas9 inhibition. In particular embodiments of the invention, an AcrIIA16Lmo, AcrIIA17Efa, AcrIIA17Sga, or AcrIIA19Ssim polypeptide is used to provide broad spectrum inhibition of multiple Cas9 proteins in vivo, ex vivo, or in vitro.

TABLE 1A
Anti-CRISPR sequences

| | | | | SEQ |
|---|---|---|---|---|
| Anti- | | | Accession | ID |
| CRISPR | Strain | ML sequence | Number | NO: |
| IIA16-Lmo | Listeria | MGYIGTKRSERSQDAIEDYEVPLNHFNKDLIQAFIDENEAYDT | WP_061665674.1 | 1 |
| | monocytogenes | LKTKKVRLWKFVAPRAGATSWHHTGTYYNKTDHYSLEKVAD | | |
| | | ELLQNGDEWEEQFKAYVKEEQETATSEPVFLSVIKVQIWGGS | | |
| | | MKRPKLVGHEVVMGVKKEGWLHAVSKATQSKYKLSANKVE | | |
| | | MQKHYSLEDYSALTKDFPEFKAQKRAINKKMKEMYN | | |
| HA16-Efa | Enterococcus | MGYVGKSRSVRSQIAIDNAEVPLNHITKDYILTFVTENNIDETL | WP_025188019.1 | 2 |
| | faecalis | KNESVAMWKFVAKRHGSTSWHHVSKHYNKIDHYDLHDVAE | | |
| | | YFSMNYDSLKNDYQNLLDQKRQAKNDLIKNLKLGIIKVQIWG | | |
| | | GTKRYPKLEGYESVMGVVKDGWLHTVTLSNQTKYKITGNKIE | | |
| | | EITIFELDQYDILTKKFPEFRAMKRKINKEVARLSK | | |
| HA17-Efa | Enterococcus | MAILNNKGEKISIDCADLISEVEEDILIFGGTFLVYAICSWREIE | WP_002401839.1 | 3 |
| | faecalis | QVEYISDYVHADNPESYKDELTTKEYAELKEIYEKDLEELKITKN | | |
| | | KQMNLNELLSILTIQNSIT | | |
| IIA17-Sga | Streptococcus | MKISVDSEKLLNEAINDFDIFGEDFNVYAIYSYREDYDFEYISDY | WP_074626943.1 | 4 |
| | gallolyticus | VDADEPTRDEFETEEDYQEVMKDFKENLDSLKFTKHKKMTIA | | |
| | | DLVHELWEQNRIF | | |
| IIA18-Sma | Streptococcus | MKIDTTVTEVKENGKTYLRLLKGNEQLKAVSDKAVAGVNLFP | WP_099390844.1 | 5 |
| | macedonicus | GAKIGSFLVRQDNIVVFPDNKGEFDLDFFNLLNDNFETLVEYA | | |
| | | KMADCLDIAFDINEKSYFNMIMWLMKNIDENWSQSPYGESF | | |
| | | YSSKDIDWGYKPEGSLRVSDHWNFGQDGEHCPTAEPVDGW | | |
| | | AVCKFENGKYHLIKKF | | |
| IIA18-Sga | Streptococcus | MKIDTTVTEVKENGKTYLRLVEGTEQLKAISDKAMAGVNLFP | WP_074627086.1 | 6 |
| | gallolyticus | GAKIDSFLVKQDSIVVFPDNKGEFDLDFFKQLDENFDTIAKYA | | |
| | | RVATCFEEVAFDEKSYFNMIMWLMDNMDENWSQSPYGES | | |
| | | FYSSKNIDWGYKPEGSLRVSDHWNFGENGEHCPTAEPVDG | | |
| | | WAVCKFENGKYHLIKKF | | |
| IIA19-Ssim | Staphylococcus | MKLIVEVEETNYKNLVNYTKLTNESHNILVNRLISEYITKPYELR | WP_107591702.1 | 7 |
| | simulans | LDLSERYSNRDLIEFKFMLIEYCKEALQDIKELANSDEAYETDEA | | |
| | | FEAVFRQLFEEVISNPDTVLKAFHSYTSFLEENK | | |
| IIA19-Spse | Staphylococcus | MKLIINIEDKNYKYLTELAQQDNTNIGSIVNNLIQTHITDVNES | WP_100006909.1 | 8 |
| | pseudintermedius | YRSVDKKELDEFSRVMQHYFHEDLASMYDVIGSDEELSTDKQ | | |
| | | MLKVYKKLYQDVALRNGIALELFNAYKKG | | |

TABLE IB
Summary of Anti-CRISPR activity

| | | | | Inhibits |
|---|---|---|---|---|
| | | Inhibits | Inhibits EfaCas9 | EfaCas9 in E. |
| | | SpCas9 in P. | in E. faecalis | faecalis native |
| | | aeruginosa | native system: | system: |
| Anti- | Inhibits | heterolo-gous | CRISPRI (Spy- | CRISPR3 (Sau- |
| CRISPR | Cas9 | system | like) | like) |
| IIA16-Lmo | Y | Y | Y | Y |
| IIA16-Efa | Y | ND | ND | ND |
| IIA17-Efa | Y | Y | Y | Y |
| IIA17-Sga | Y | Y | Y | Y |
| IIA18-Sma | Y | Y | ND | ND |
| IIA18-Sga | Y | ND | ND | ND |
| IIA19-Ssim | Y | Y | Y | Y |
| IIA19-Spse | Y | ND | ND | ND |

ND: Not determined

### 4. Introduction into Cells

The present disclosure provides methods of inhibiting a Cas9-polypeptide in a cell, comprising introducing any of the herein-described Cas9-inhibiting polypeptides into the cell, wherein the Cas9-inhibiting polypeptide is heterologous to the cell and is substantially (e.g., at least about 60%, 70%, 80%, 90%, 95%) identical to any one or more of the sequences shown as SEQ ID NOS: 1-8, or a fragment thereof. In some embodiments, the Cas9-inhibiting polypeptide comprises a sequence selected from SEQ ID NOS: 1-8, or a fragment thereof. In some embodiments, the polypeptide comprises a sequence selected from the group consisting of SEQ ID NO: 1, 2, 4, and 7. In some embodiments, the Cas9-inhibiting polypeptide can inhibit one or more Cas9-inhibiting polypeptides selected from the group consisting of SpyCas9, Efa1Cas9, and Efa3Cas9.

The Cas9-inhibiting polypeptides can be introduced into any prokaryotic or eukaryotic cell to inhibit Cas9 in that cell. In some embodiments, the cell contains Cas9 protein when the Cas9-inhibiting polypeptide is introduced into the cell. In other embodiments, the Cas9-inhibiting polypeptide is introduced into the cell and then Cas9 polypeptide is introduced into the cell.

Introduction of the Cas9-inhibiting polypeptides into the cell can take different forms. For example, in some embodiments, the Cas9-inhibiting polypeptides themselves are introduced into the cells. Any method for introduction of polypeptides into cells can be used. For example, in some embodiments, electroporation, or liposomal or nanoparticle delivery to the cells can be employed. In other embodiments, a polynucleotide encoding a Cas9-inhibiting polypeptide is introduced into the cell and the Cas9-inhibiting polypeptide is subsequently expressed in the cell. In some embodiments, the polynucleotide is an RNA. In some embodiments, the polynucleotide is a DNA.

In some embodiments, the Cas9-inhibiting polypeptide is expressed in the cell from RNA encoded by an expression cassette, wherein the expression cassette comprises a promoter operably linked to a polynucleotide encoding the Cas9-inhibiting polypeptide. In some embodiments, the promoter is heterologous to the polynucleotide encoding the Cas9-inhibiting polypeptide. Selection of the promoter will depend on the cell in which it is to be expressed and the desired expression pattern. In some embodiments, promoters are inducible or repressible, such that expression of a nucleic acid operably linked to the promoter can be expressed under selected conditions. In some examples, a promoter is an inducible promoter, such that expression of a nucleic acid operably linked to the promoter is activated or increased. Accordingly, the present disclosure provides expression cassettes comprising a polynucleotide encoding any of the herein-described Cas9-inhibiting proteins, operably linked to a promoter.

An inducible promoter may be activated by the presence or absence of a particular molecule, for example, doxycycline, tetracycline, metal ions, alcohol, or steroid compounds. In some embodiments, an inducible promoter is a promoter that is activated by environmental conditions, for example, light or temperature. In further examples, the promoter is a repressible promoter such that expression of a nucleic acid operably linked to the promoter can be reduced to low or undetectable levels, or eliminated. A repressible promoter may be repressed by direct binding of a repressor molecule (such as binding of the trp repressor to the trp operator in the presence of tryptophan). In a particular example, a repressible promoter is a tetracycline repressible promoter. In other examples, a repressible promoter is a promoter that is repressible by environmental conditions, such as hypoxia or exposure to metal ions.

In some embodiments, the polynucleotide encoding the Cas9-inhibiting polypeptide (e.g., as part of an expression cassette) is delivered to the cell by a vector. For example, in some embodiments, the vector is a viral vector. Exemplary viral vectors can include, but are not limited to, adenoviral vectors, adeno-associated viral (AAV) vectors, and lentiviral vectors. Accordingly, the present disclosure provides vectors comprising any of the herein-described polynucleotides or expression vectors.

In some embodiments, the Cas9-inhibiting polypeptide or a polynucleotide encoding the Cas9-inhibiting polypeptide is delivered as part of or within a cell delivery system. Various delivery systems are known and can be used to administer a composition of the present disclosure, for example, encapsulation in liposomes, microparticles, microcapsules, or receptor-mediated delivery.

Exemplary liposomal delivery methodologies are described in Metselaar et al., Mini Rev. Med Chem. 2(4):319-29 (2002); O'Hagen et al., Expert Rev. Vaccines 2(2):269-83 (2003); O'Hagan, Curr. Drug Targets Infect. Disord. 1(3):273-86 (2001); Zho et al., Biosci Rep. 22(2):355-69 (2002); Chikh et al., Biosci Rep. 22(2):339-53 (2002); Bungener et al., Biosci. Rep. 22(2):323-38 (2002); Park, Biosci Rep. 22(2):267-81 (2002); Ulrich, Biosci. Rep. 22(2):129-50; Lofthouse, Adv. Drug Deliv. Rev. 54(6):863-70 (2002); Zhou et al., J. Inmunmunother. 25(4):289-303 (2002); Singh et al., Pharm Res. 19(6):715-28 (2002); Wong et al., Curr. Med. Chem. 8(9):1123-36 (2001); and Zhou et al., Immunonmethods (3):229-35 (1994).

Exemplary nanoparticle delivery methodologies, including gold, iron oxide, titanium, hydrogel, and calcium phosphate nanoparticle delivery methodologies, are described in Wagner and Bhaduri, Tissue Engineering 18(1): 1-14 (2012) (describing inorganic nanoparticles); Ding et al., Mol Ther e-pub (2014) (describing gold nanoparticles); Zhang et al., Langmuir 30(3):839-45 (2014) (describing titanium dioxide nanoparticles); Xie et al., Curr Pharm Biotechnol 14(10):918-25 (2014) (describing biodegradable calcium phosphate nanoparticles); and Sizovs et al., J Am Chem Soc 136(1):234-40 (2014).

Introduction of a Cas9-inhibiting polypeptide as described herein into a prokaryotic cell can be achieved by any method used to introduce protein or nucleic acids into a prokaryote. In some embodiments, the Cas9-inhibiting polypeptide is delivered to the prokaryotic cell by a delivery vector (e.g., a bacteriophage) that delivers a polynucleotide encoding the Cas9-inhibiting polypeptide. In some embodiments, inhibiting Cas9 in the prokaryote using a Cas9-inhibiting polypeptide of the invention could either help the phage kill the bacterium or help other phages kill it. In some embodiments, the Cas9-inhibiting polypeptide is introduced by a bacteriophage in the context of phage therapeutics, i.e., the use of bacteriophage to treat pathogenic bacterial infections, and the Cas9-inhibiting polypeptide increases the potency of the bacteriophage by inhibiting Cas9 present in the targeted bacteria.

### 5. Cells

A Cas9-inhibiting polypeptide as described herein can be introduced into any cell that contains, expresses, or is expected to express, Cas9. Exemplary cells can be prokaryotic or eukaryotic cells. Exemplary prokaryotic cells can include but are not limited to, those used for biotechnological purposes, the production of desired metabolites, E. coli and human pathogens. Examples of such prokaryotic cells can include, for example, Escherichia coli, Pseudomonas sp., Corynebacterium sp., Bacillus subtitis, Streptococcus pneumonia, Pseudomonas aeruginosa, Staphylococcus aureus, Campylobacter jejuni, Francisella novicida, Corynebacterium diphtheria, Enterococcus sp., Listeria monocytogenes, Mycoplasma gallisepticum, Streptococcus sp., or Treponema denticola. Exemplary eukaryotic cells can include, for example, animal (e.g., mammalian) or plant cells. Exemplary mammalian cells include but are not limited to human, non-human primates. mouse, and rat cells. Cells can be cultured cells or primary cells. Exemplary cell types can include, but are not limited to, induced pluripotent cells, stem cells or progenitor cells, and blood cells, including but not limited to T-cells or B-cells. Accordingly, the present disclosure provides cells comprising any of the herein-described Cas9-inhibiting polypeptides, polynucleotides expression cassettes, or vectors

In some embodiments, the cells are infectious prokaryotic pathogens that possess functional CRISPR-Cas9, and the Cas9-inhibiting polypeptide is introduced to reduce the virulence of the pathogen. In some embodiments, the infectious pathogens are targeted with bacteriophage, and the Cas9-inhibiting polypeptide is introduced together with the phage to enhance the potency of the phage against the pathogen.

In some embodiments, the cells are removed from an animal (e.g., a human, optionally in need of genetic repair), and then Cas9, and optionally guide RNAs, for gene editing are introduced into the cell ex vivo, and a Cas9-inhibiting polypeptide is introduced into the cell. In some embodiments, the cell(s) is subsequently introduced into the same animal (autologous) or different animal (allogeneic).

In any of the embodiments described herein, a Cas9 polypeptide can be introduced into a cell to allow for Cas9 DNA binding and/or cleaving (and optionally editing), followed by introduction of a Cas9-inhibiting polypeptide as described herein. This timing of the presence of active Cas9 in the cell can thus be controlled by subsequently supplying Cas9-inhibiting polypeptides to the cell, thereby inactivating Cas9. This can be useful, for example, to reduce Cas9 “off-target” effects such that non-targeted chromosomal sequences are bound or altered. By limiting Cas9 activity to a limited “burst” that is ended upon introduction of the Cas9-inhibiting polypeptide, one can limit off-target effects. In some embodiments, the Cas9 polypeptide and the Cas9-inhibiting polypeptide are expressed from different inducible promoters, regulated by different inducers. These embodiments allow for first initiating expression of the Cas9 polypeptide followed by induction of the Cas9-inhibiting polypeptide, optionally while removing the inducer of Cas9 expression.

In some embodiments, a Cas9-inhibiting polypeptide as described herein can be introduced (e.g., administered) to an animal (e.g., a human) or plant. This can be used to control in vivo Cas9 activity, for example in situations in which CRISPR-Cas9 gene editing was performed in vivo, or in circumstances in which an individual is exposed to unwanted Cas9, for example where a bioweapon comprising Cas9 is released.

In some embodiments, a Cas9-inhibiting polypeptide as described herein can be introduced to an animal (e.g., an insect), plant, or fungus in the context of limiting the extent of a gene drive. Gene drives involve the propagation of a gene or genes through a population or species by increasing the probability that a specific allele or alleles will be transmitted to progeny. CRISPR-Cas9 can be used in gene drives, in which an integrated construct comprises the specific allele that is being propagated and comprises a guide RNA and Cas9 that enable the targeted cleavage of a homologous locus in a cell and the CRISPR-mediated transfer of the specific allele to the homologous locus. Cas9-inhibiting polypeptides could be used, e.g., to protect specific subpopulations or individuals from the effects of a gene drive, or to slow or stop the spread of a gene drive throughout a population.

Any of a large spectrum of Cas9 proteins can be inhibited by the present Cas9-inhibiting polypeptides. For example, Cas9 from Streptococcus pyogenes, Staphylococcus aureus, Neisseria meningitidis, Campylobacter jejuni, Francisella novicida, Streptococcus thermophiles, and others can be inhibited.

### 6. Compositions

In some embodiments, a Cas9-inhibiting polypeptides as described herein or a polynucleotide encoding a Cas9-inhibiting polypeptide as described herein, is administered as a pharmaceutical composition. Accordingly, in some embodiments, the present disclosure provides a composition comprising any of the herein-described Cas9-inhibiting polypeptides or polynucleotides encoding any of the herein-described Cas9-inhibiting polypeptide, and a pharmaceutically acceptable carrier. In some embodiments, the present disclosure provides a delivery such as a liposome, nanoparticle or other delivery vehicle as described herein or otherwise known, comprising any of the herein-described Cas9-inhibiting polypeptides or a polynucleotide encoding any of the herein-described Cas9-inhibiting polypeptides. The compositions can be administered directly to a mammal (e.g., human) to inhibit Cas9 using any route known in the art, including e.g., by injection (e.g., intravenous, intraperitoneal, subcutaneous, intramuscular, or intrademal), inhalation, transdermal application, rectal administration, or oral administration.

The pharmaceutical compositions of the invention may comprise a pharmaceutically acceptable carrier. Pharmaceutically acceptable carriers are determined in part by the particular composition being administered, as well as by the particular method used to administer the composition. Accordingly, there are a wide variety of suitable formulations of pharmaceutical compositions of the present invention (see, e.g., Remington's Pharmaceutical Sciences, 17th ed., 1989).

### 7. Examples

The following examples are offered to illustrate, but not to limit, the claimed invention.

### Example 1: Broad-Spectrum Anti-CRISPR Proteins Facilitate Horizontal Gene Transfer

### Results

### Novel Type II-A Anti-CRISPRs (AcrIIA16-19) Block SpyCas9 Binding to DNA

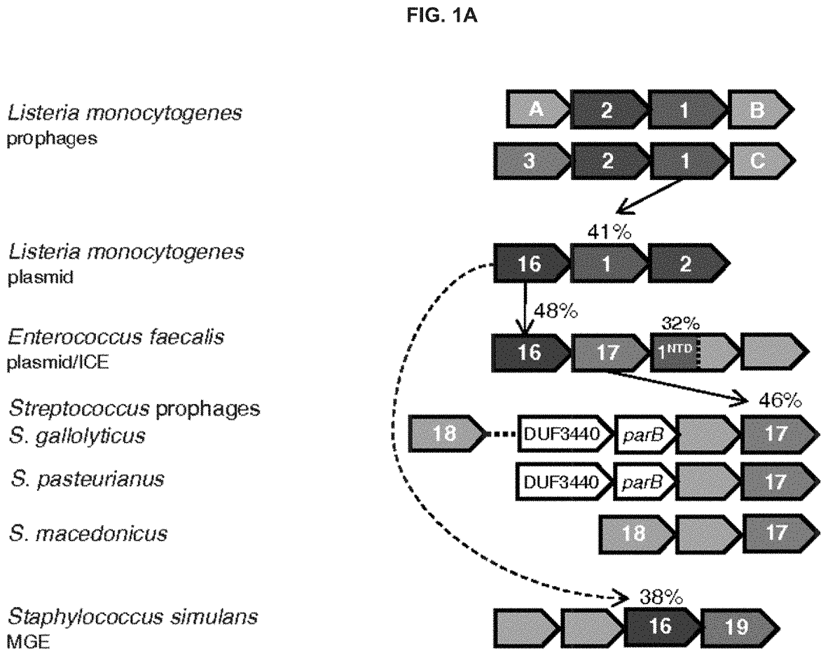

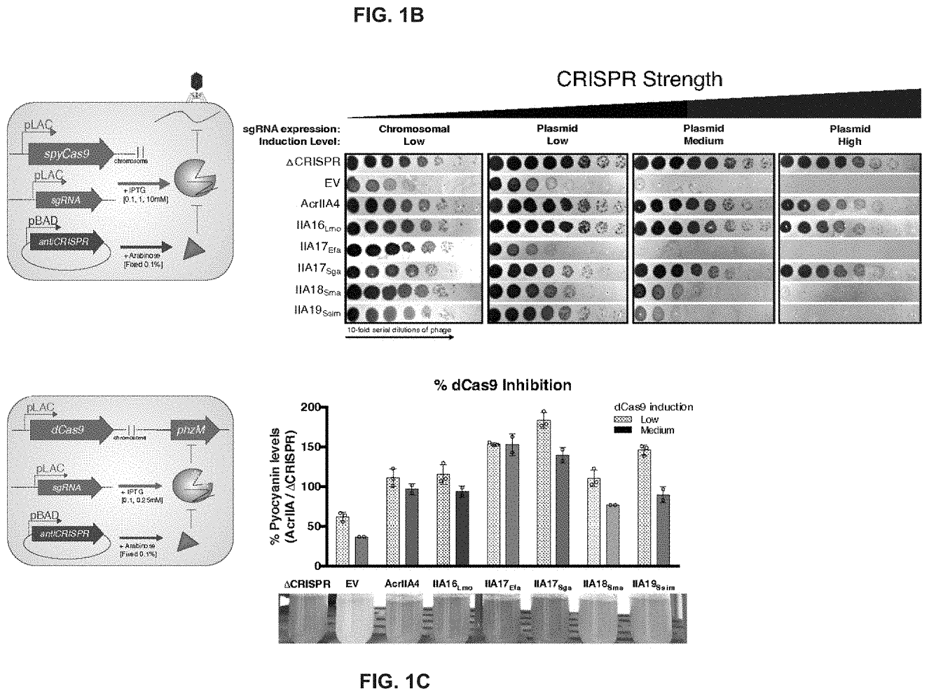

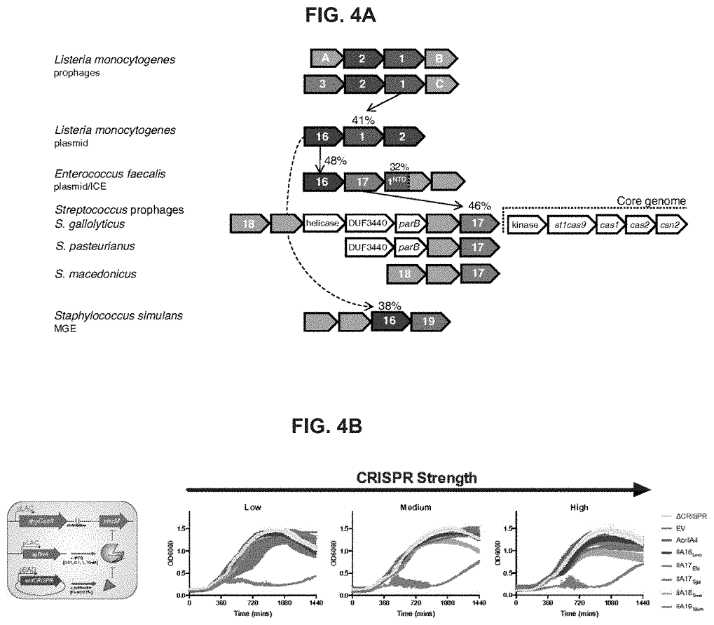

To identify undiscovered acr genes, we first utilized the widespread acrIIA1 gene as an anchor in bioinformatic searches across genomes on NCBI (FIG. 1A). An AcrIIA1 homolog (41% amino acid sequence identity) was previously identified within an L. monocytogenes plasmid, along with an AcrIIA2 homolog that was recently characterized (AcrIIA2b.3, Jiang et al., 2019). Genomic neighbors in this locus were tested against the Type II-A Cas9 system using a SpyCas9 phage-targeting screening system in Pseudomonas aeruginosa (FIG. 1B; Borges et al., 2018; Jiang et al., 2019). Gene AWI79_RS12835 (now acrIIA16) inhibited SpyCas9 in this assay. Similarly, using acrIIA16 as the anchor gene, functional analysis of its neighbors revealed three more distinct anti-CRISPR genes (acrIIA17-19) identified in Enterococcus, Streptococcus, and Staphylococcus (FIG. 1A). To quantify the strength of SpyCas9 inhibition, Cas9 and the sgRNA were titrated via IPTG induction. At the lowest CRISPR-Cas expression level, all identified acrIIA genes inhibited SpyCas9, restoring phage replication to nearly the same levels as in the strain lacking CRISPR immunity (ΔCRISPR, FIG. 1B). However, at higher CRISPR-Cas expression levels, only AcrIIA16Lmo, AcrIIA17Sga, and control AcrIIA4 maintained inhibition against SpyCas9 (FIG. 1B). In agreement with this result, the AcrIIA proteins also protect against self-genome cleavage assay with similar strength (FIG. 4B).

To inspect the mechanism of these new AcrIIA proteins, we established a CRISPRi assay, where catalytically dead SpyCas9 (dCas9) is programmed to bind the promoter of the phzM gene. Repression of phzM halts the production of green pigment called pyocyanin, generating a yellow culture (Bondy-Denomy et al., 2015). In the presence of AcrIIA4, DNA binding by dCas9 is inhibited generating a green culture. AcrIIA16-19 all presented a similar phenotype at two dCas9 induction levels, suggesting that these new AcrIIAs inhibit SpyCas9 at the step of target DNA binding or another upstream stage (FIG. 1C).

acrIIA Genes Protect Plasmids from CRISPR-Targeting During Conjugation

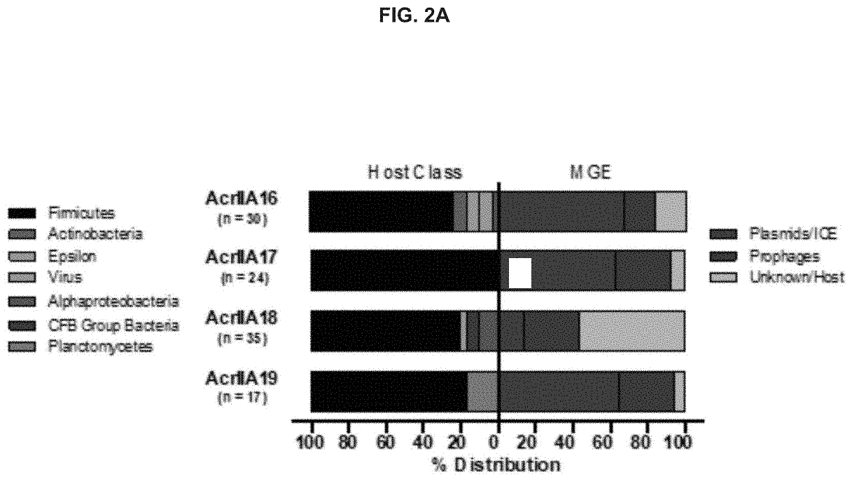

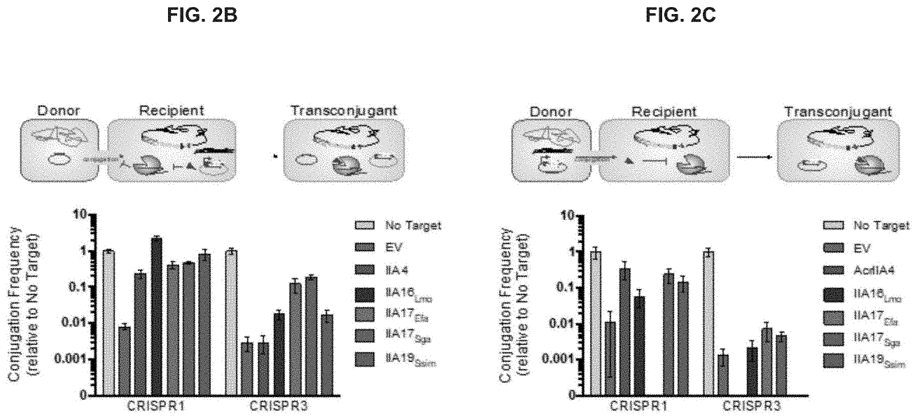

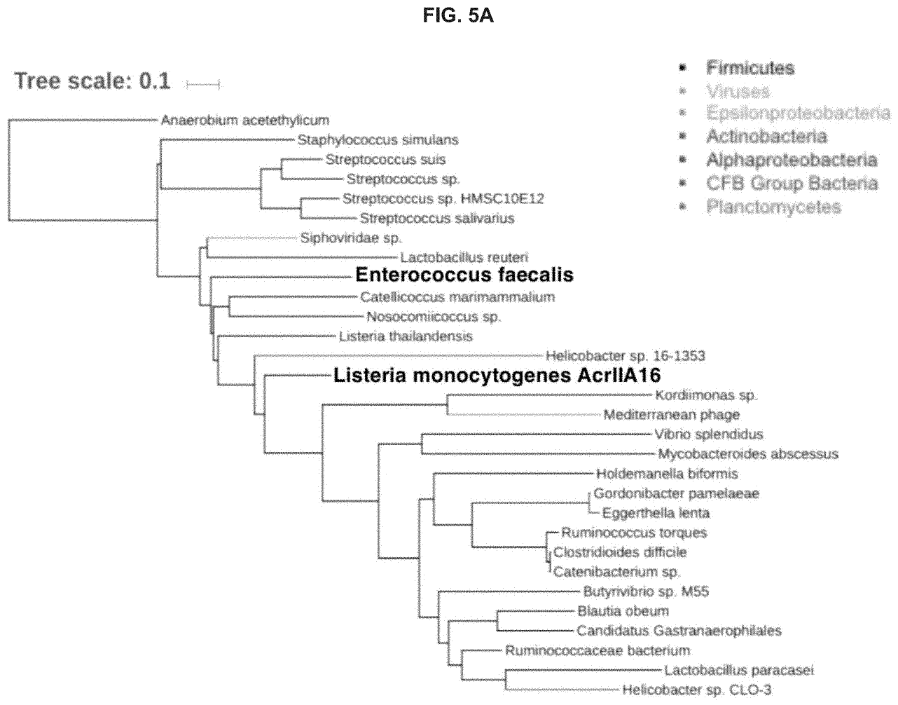

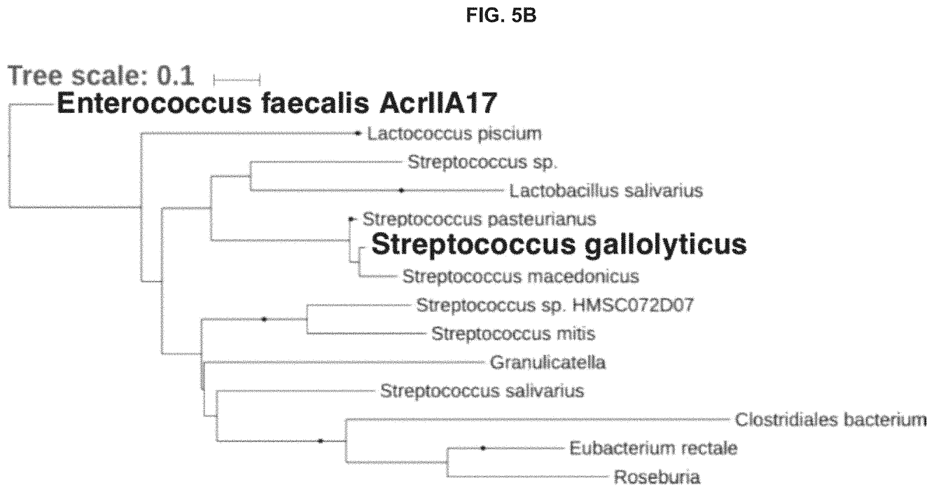

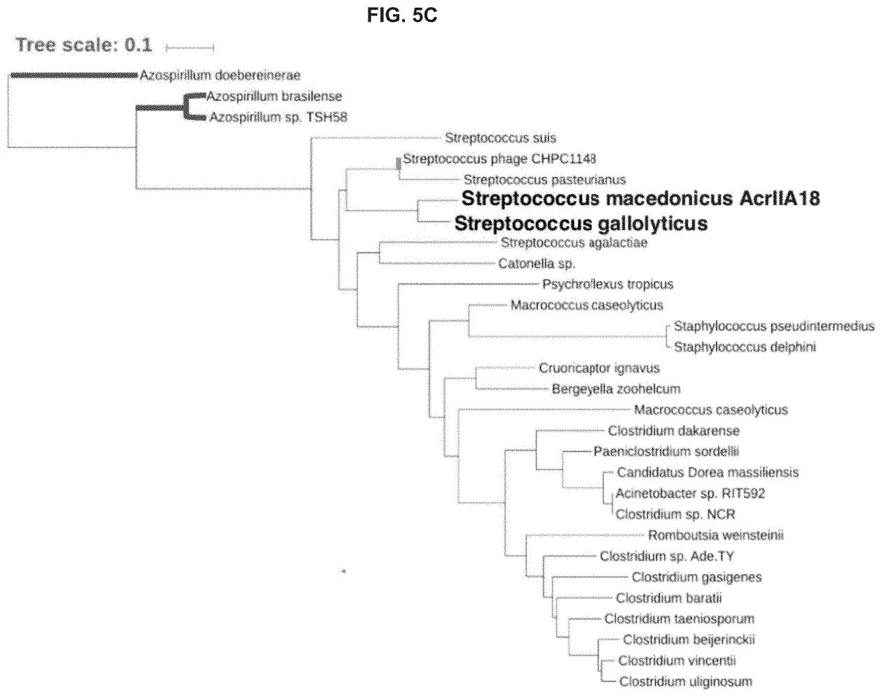

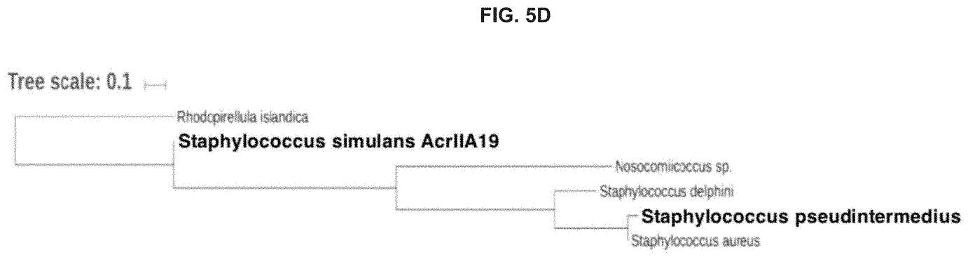

Analysis of AcrIIA16-19 distribution revealed that most orthologs are present in diverse conjugative MGEs of Firmicutes, with only a small percentage found in phages and other bacterial classes (FIG. 2A, FIG. 5). Genes adjacent to the acr loci were examined for presence of prophage or plasmid genes. Interestingly, acrIIA16, 17, and 19 exist primarily in non-phage MGEs including plasmids or ICEs. We reasoned that these Acr proteins could be suited to offer protection to conjugative elements (FIG. 2A).

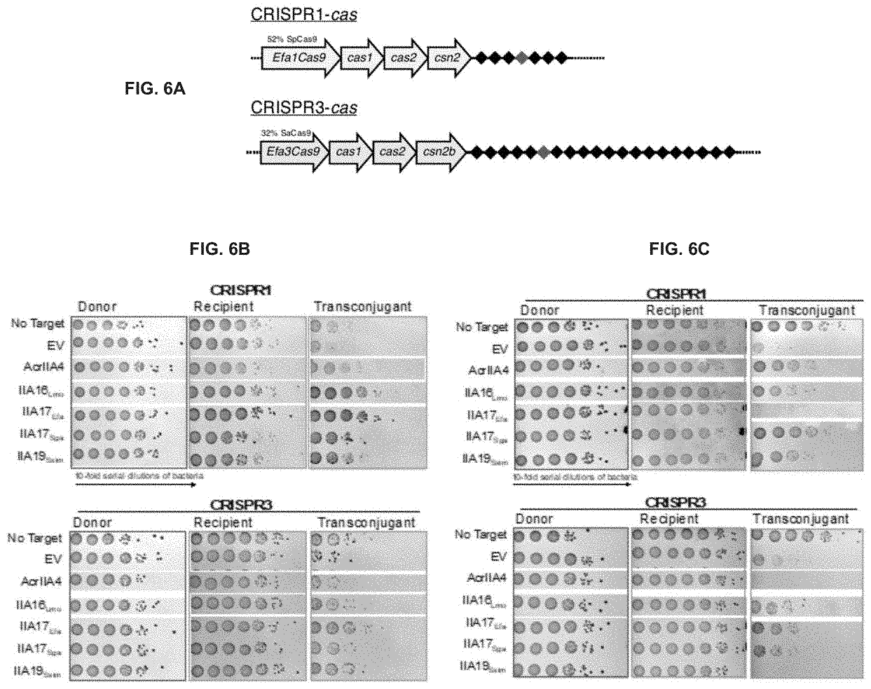

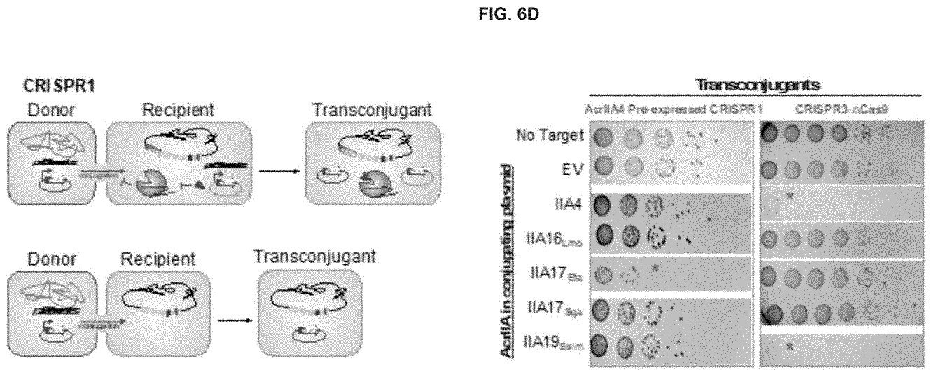

To investigate AcrIIA activity during plasmid conjugation, we tested the ability of Cas9 to target a plasmid when an AcrIIA protein is expressed either in the recipient or by the conjugating element. Conjugation efficiency (and thus, Cas9 targeting efficiency) was assessed through an antibiotic resistance marker on the conjugative element. Previously reported E. faecalis strains (Hullahalli et al., 2017) were used for this assay, with acrIIA genes individually expressed from an E. faecalis promoter native to the acr locus. E. faecalis encodes two distinct endogenous Type II-A CRISPR-Cas variants—CRISPR1, which is 52% identical to SpyCas9 and CRISPR3, which is ˜32% identical to SauCas9 (FIG. 6A). When acrIIA16, 17, and 19 were pre-expressed in recipient cells, all inhibited CRISPR1 robustly, and CRISPR3 to a lesser degree (FIGS. 2B, 6B). acrIIA4 only inhibited CRISPR1 activity, which encodes a Cas9 that has a similar PAM-interacting domain to SpyCas9 (FIG. 2B).

We then sought to investigate whether AcrIIA proteins could function during plasmid conjugation when acrIIA genes were expressed from the conjugating CRISPR-targeted plasmid. acrIIA16-17 and acrIIA19 were indeed protective against CRISPR1 plasmid targeting when produced during conjugation, while acrIIA17 orthologs provided modest protection against CRISPR3 (FIGS. 2C, 6C). Oddly, plasmids expressing certain acr genes did not produce detectable transconjugants (e.g. acrIIA17Efa when challenged with CRISPR1 and acrIIA4/acrIIA19Ssim against CRISPR3), but this was independent of CRISPR-targeting (FIG. 6D), for a reason that is unknown. We conclude that acrIIA genes are able to inhibit both CRISPR-Cas9 systems during plasmid conjugation in E. faecalis and can enhance HGT by >1 order of magnitude when pre-expressed in recipient cells.

AcrIIA16-19 Proteins Interact with SpyCas9

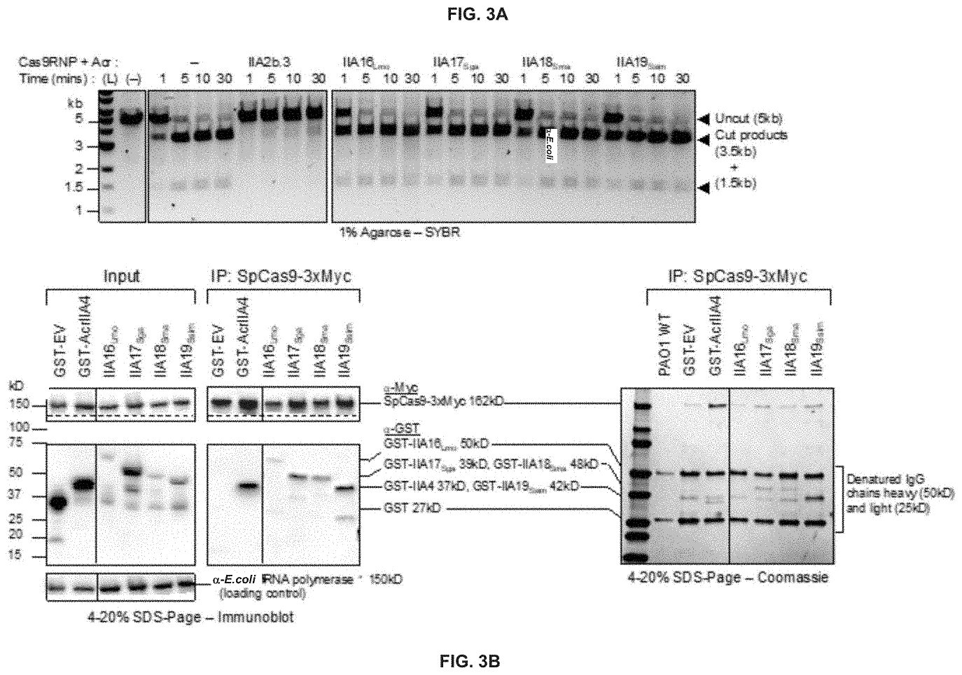

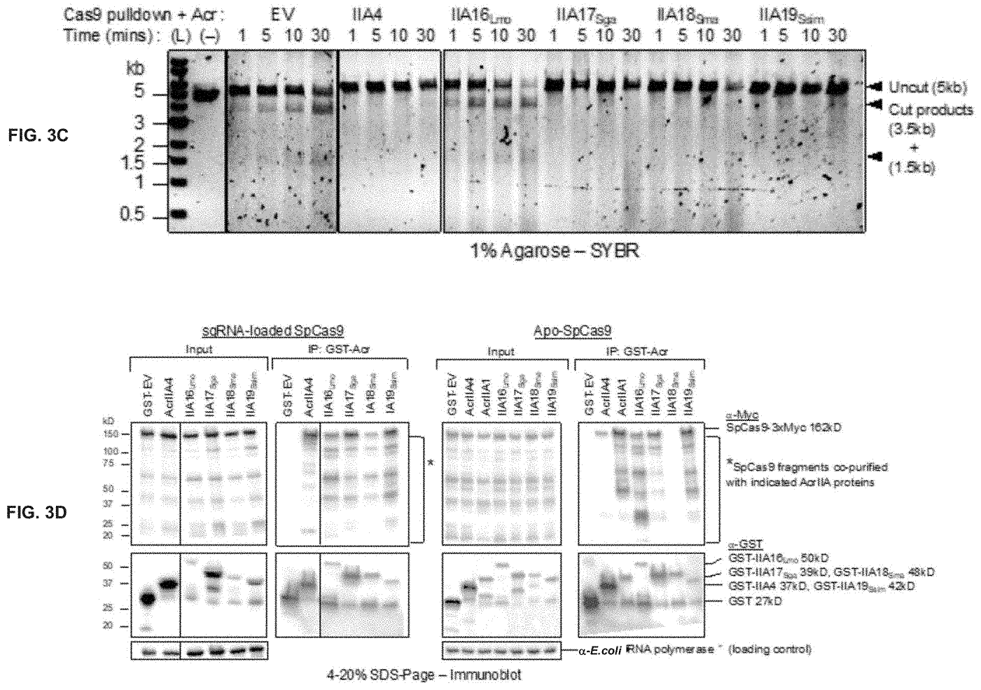

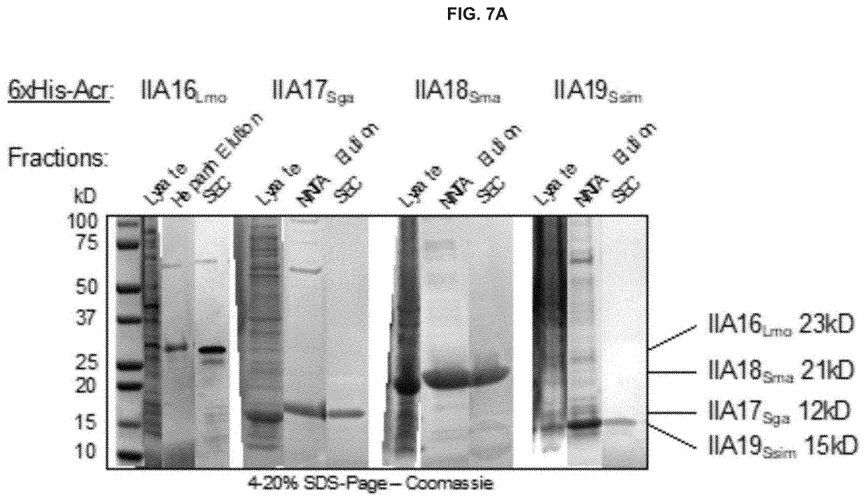

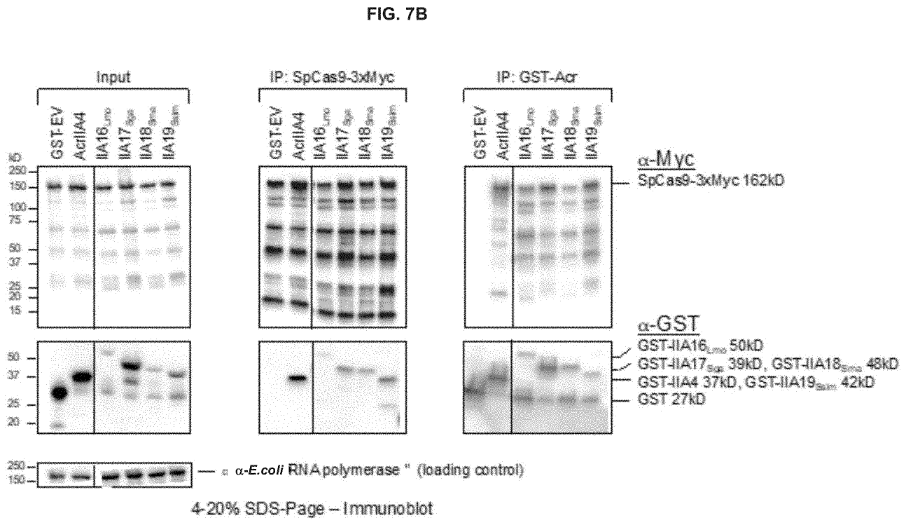

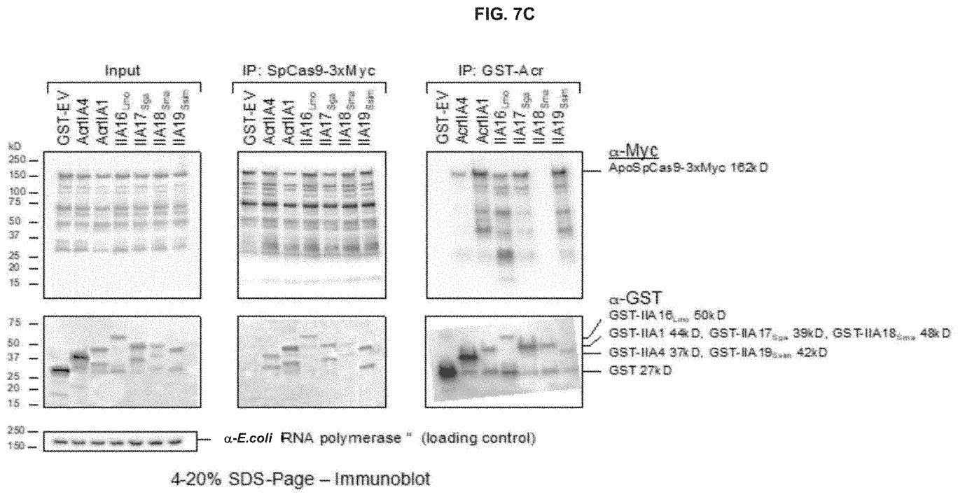

To further investigate the mechanism of inhibition of the new AcrIIAs, we purified one homolog of AcrIIA16-19 to directly test their effect on SpyCas9 activity (FIG. 7A). In vitro cleavage experiments revealed that purified AcrIIA16-19 proteins do not inhibit SpyCas9-mediated DNA cleavage, while the positive control AcrIIA2b.3 does (FIG. 3A). Given that all the AcrIIA purified proteins did not inhibit SpyCas9 activity in vitro, we considered that the cellular environment may be essential for their function. Immunoprecipitation of SpyCas9 from bacteria co-expressing each AcrIIA protein demonstrated that AcrIIA16-19 interact with SpyCas9-sgRNA (FIG. 3B). The absence of any other stoichiometric, co-purifying proteins suggests a direct interaction between Cas9 and the Acr proteins (FIG. 3B, right gel). Interestingly, SpyCas9 co-purified with AcrIIA17-19 does not perform DNA cleavage, although SpyCas9 co-purified with AcrIIA16Lmo is not inhibited (FIG. 3C). The failure of AcrIIA16Lmo to inhibit SpyCas9 in vitro is likely due to its low expression level, as visualized in the input western blot (FIG. 3B).

In conducting the immunoprecipitation experiments (above), we noticed that SpyCas9 expressed in our strain of Pseudomonas aeruginosa exhibited a series of degradation products when blotted for the C-terminal Myc tag (FIG. 3D). Upon closer inspection of the GST-Acr pulldowns, enriched SpyCas9 fragments co-immunoprecipitated with AcrIIA16-19 appear to be different from those of AcrIIA4, suggesting a distinct binding mechanism. To test this, we immunoprecipitated AcrIIA16-19 from P. aeruginosa expressing Apo-SpyCas9 without sgRNA, a complex previously reported to be only a weak AcrIIA4 binding partner (Shin et al., 2017). AcrIIA16-17 and AcrIIA19 co-purified with Apo-SpyCas9, while AcrIIA4 shows weak binding (comparing the relative amount of AcrIIA4 to Cas9). Interestingly, AcrIIA18 does not appear to interact with Apo-SpyCas9 (FIGS. 3D, 7C). These results suggest that AcrIIA16, 17, and 19 have distinctive SpyCas9 interacting sites from AcrIIA4, with AcrIIA18 additionally displaying a unique binding profile.

### Discussion

Numerous strategies continue to be developed for identification of Acrs, with a remarkably diverse range of disclosed inhibition mechanisms. Here, we employed a “guilt-by-association” bioinformatics approach to discover new acr genes in various MGEs. Given the reported coexistence of acrIIA1 with other acrs, it is an effective anchor gene to utilize in searches of acr loci (Rauch et al., 2017; Jiang et al., 2019; Osuna et al., 2019). The acr genes reported here are found in plasmids and ICEs, as well as some prophages, and other uncharacterized elements. These Cas9 inhibitors successfully protect phage DNA during infection and plasmid DNA during conjugation. AcrIIA16-19 interact with SpyCas9 via novel binding mechanisms compared to AcrIIA4 and AcrIIA2, to inhibit target DNA binding and cleavage in vitro and in vivo. Finally, the new AcrIIA proteins, e.g., AcrIIA16Lmo, AcrIIA16Efa, AcrIIA17Sga, and AcrIIA19Ssim, displayed broad-spectrum inhibition of Type II-A Cas9 orthologs.

It is of high clinical relevance to find acrIIA genes in E. faecalis, where the spread of antibiotic resistance genes is frequently promoted through plasmid transfer despite the presence of host-encoded CRISPR-Cas systems. This work opens the door to the identification of more acr genes in this organism. Previous work has shown that multidrug resistant E. faecalis strains are more likely to lack CRISPR-Cas9 but can acquire MGEs with protospacer matches due to low levels of Cas9 expression, and tolerate those plasmids transiently (Palmer and Gilmore, 2010; Hullahalli et al. 2017; Hullahalli et al. 2018). Our results suggest that these complex interactions have an additional layer and that a state of plasmid self-targeting could be stabilized for some time prior to potential CRISPR-Cas or spacer loss. We demonstrated that AcrIIA proteins not only could enhance the spread of a given antibiotic resistance plasmid, but it also limits the hosts ability to limit the acquisition of other MGEs.

With the increasing use of CRISPR-Cas systems for various genome editing applications, the discovery and characterization of natural inhibitors that regulate a variety of Cas9 orthologs via different mechanisms remains critical. The broad-spectrum inhibitors are attractive as practical regulators of multiple distinct Cas9 proteins.

### Methods

### Microbes

Escherichia coli (DH5α, XL1Blue, NEB 10-beta, or NEB turbo) were routinely cultured in lysogeny broth (LB) at 37° C. supplemented with antibiotics at the following concentrations: gentamicin (30 μg/mL), carbenicillin (100 μg/mL), kanamycin (25 μg/mL), chloramphenicol (25 μg/mL), erythromycin (300 μg/mL) or tetracycline (10 μg/mL). Pseudomonas aeruginosa (PAO1) was cultured in LB medium at 37° C. with supplemented antibiotics for plasmid maintenance: gentamicin (50 μg/mL) or carbenicillin (250 μg/mL). For maintaining multiple plasmids in the same P. aeruginosa strain, antibiotic concentrations were adjusted to 30 μg/mL gentamicin and 100 μg/mL carbenicillin. All Enterococcus faecalis strains (C173, OG1RF, T11RF, T11RFΔCas9) were cultured in brain-heart-infusion (BHI) medium at 37° C., unless otherwise mentioned. Antibiotics were used in the following concentrations: spectinomycin (500 μg/mL), streptomycin (500 μg/mL), rifampicin (50 μg/mL), fusidic acid (25 μg/mL), chloramphenicol (15 μg/mL) or erythromycin (50 μg/mL).

Construction of P. aeruginosa and E. faecalis Strains

P. aeruginosa heterologous type II-A system was generated as previously described (Borges et al., 2018) under “construction of PAO1::SpyCas9 expression strain,” with sgRNA integrated into the bacterial genome using the mini-CTX2 vector (Hoang et al., 2000) or expressed from multi-copy episomal plasmid pMMB67HE-PLac for in vivo assays, and plasmid pHERD30T-PBad for in vitro assays. All acr candidate genes were synthesized as gene fragments (Twist Biosciences) and cloned using Gibson Assembly into plasmids of P. aeruginosa vectors pHERD30T or pMMB67HE, and E. faecalis vectors pKH12 or pMSP3535 (gifts from Kelli L. Palmer and Gary Dunny RRID:Addgene_46886 respectively). Plasmids were electroporated into PAO1 (Choi et al., 2006) for all P. aeruginosa strains, and E. faecalis strains C173, OG1RF, T11RF and T11RFΔCas9 using previously published protocols (Bhardwaj et al., 2016). All strains and plasmids constructed and used in this study are listed in Table 2.

Bacteriophage Plaque Assays in P. aeruginosa

Plaque assays were performed as previously described (Borges et al, 2018; Jiang et al. 2019) with sgRNA designed to target Pseudomonas phage JBD30. The PLac promoter driving chromosomally integrated SpyCas9 and sgRNA, or pMMB67HE-sgRNA was induced with titrating levels of IPTG (0.1, 1, 10 mM) and the PBad promoter driving pHERD30T-acr with 0.1% arabinose. One representative plate for each candidate were imaged using Gel Doc EZ Gel Documentation System (Bio-Rad) and Image Lab software.

Self-Genome Targeting and CRISPRi Assay in P. aeruginosa

Strains with chromosomally integrated WT SpyCas9 or dCas9 are programmed with pMMB67HE-sgRNA to target the PAO1 chromosomal phzM gene promoter in the presence of pHERD30T-acr. Cultures were grown overnight in LB supplemented with appropriate antibiotics for plasmid maintenance and 0.1% arabinose to pre-induce anti-CRISPR expression. Overnight cultures are diluted in 1:100 LB supplemented with inducers 0.1% arabinose and IPTG (0.01, 0.1, 0.25, 1, 10 mM to titrate CRISPR strength) in a 96-well Costar plate (150 μL/well) for self-targeting survival analysis or glass tubes (3 mL) for CRISPRi, in triplicates. Self-genome targeting was assayed by measuring bacterial growth curves for 16-24 hours in Synergy H1 microplate reader (BioTek, using Gen5 software) at 37° C. with continuous shaking, and data displayed as the mean OD600 of at least three biological replicates±standard deviation (error bars) as a function of time. For CRISPRi, cells were grown for 20-24 hours with continuous shaking. Next, pyocyanin was extracted and quantified as previously described (Bondy-Denomy et al., 2015). Data are displayed as the mean OD520 of at least three biological replicates±standard deviation (error bars) and representative pictures are shown.

Conjugation Assay in E. faecalis

Protospacers perfectly matching to indicated spacers in CRISPR1 or CRISPR3 array (FIG. 6A) were synthesized as complementary oligonucleotides (IDT) and cloned into pKH12 (Hullahalli et al., 2017) to generate the targeted conjugative plasmid. The promoter region of the of acr loci in E. faecalis (nucleotide sequence 350 bp upstream) was synthesized (Twist Bioscience) and cloned upstream the acr genes of the targeted pKH12 conjugative plasmid or pMSP3535. The derivatives of pKH12 were introduced into the C173 donor strain as the transferring plasmid, and pMSP3535 into OG1RF, T11RF or T11RFΔCas9 to pre-express the Acr proteins in recipient cells.

Conjugation mating experiments were performed as described by Price et al., 2016, except for the following adjustments. Diluted cultures of plasmid-donor and recipient strains were grown to OD600 0.9-1.0, after which 100 μL of donor strain was mixed with 900 μL of OG1RF recipient strains or 500 μL donor with 500 μL of T11RF recipients. Resuspended pellets were plated on Mixed Cellulose Ester filter membranes (Advantec #A020H047A) on BHI agar plates without selection and incubated overnight at 37° C. The next day, mated cells were collected by washing the filter membrane with 1.5 mL of 1×PBS and 10-fold serial dilutions were plated or spotted on BHI agar plates supplemented with antibiotics to quantify donor (spectinomycin, streptomycin and chloramphenicol), recipient (rifampicin and fusidic acid, and erythromycin for pMSP353 containing strains) or transconjugant (rifampicin, fusidic acid and chloramphenicol, with erythromycin for pre-expressed Acr strains) populations. Plates were incubated for 48 to 72 hours at 30° C. to allow colonies to develop. Plates with 30 to 300 colonies were used to calculate CFU/mL and conjugation frequency was determined by dividing the number of transconjugants over donors. For plates with spotted dilutions, the fold reductions in transconjugants were qualitatively derived by examining at least three replicates of each experiment. Plate images were acquired as above in the section “bacteriophage plaque assays in P. aeruginosa” and a representative picture is shown.

### Expression and Purification of Anti-CRISPR Proteins

N-terminally 6×His-tagged (SEQ ID NO: 9) Acr proteins were purified from E. coli BL21 following the protocol in Osuna et al., 2019 under “Cas9 and anti-CRISPR protein expression and purification”. AcrIIA16 lysate was incubated with HiTrap Heparin HP affinity column (GE #17040601), while AcrIIA2b.3, IIA17, IIA18 and IIA19 were incubated with Ni-NTA Agarose Beads (Qiagen). All elutions were dialyzed by SEC using ENrich SEC 650 10×300 Column (Bio-Rad #780-1650) to remove imidazole.

### Cleavage Assays Using Purified Proteins

Lyophilized crRNA was resuspended, complexed with tracrRNA in Nuclease-free Duplex Buffer following protocol from IDT, and incubated with SpyCas9 (NEB) at room temperature for 15 mins to form SpyCas9-RNP. All reactions were carried out in 1×MST Buffer (50 mM Tris-Cl pH 7.4, 150 mM NaCl, 20 mM MgCl2, 5 mM DTT, 5% Glycerol, 0.05% Tween-20 [v/v]). 25 nM SpyCas9-RNP was incubated with 250 nM of Acr protein for 1 h on ice. DNA substrate linearized by NheI digestion was added to a final concentration of 2 nM and the reaction was allowed to cut for 0, 5, 10 and 30 mins, at each timepoint the reaction was quenched in warm Quench Buffer (50 mM EDTA, 0.02% SDS) followed by heating at 95° C. for 10 mins. Products were analyzed on 1% agarose gel and stained with SYBR Safe.

### Co-Immunoprecipitation of SpyCas9-3xMyc and GST-Acr

Chromosomally integrated SpyCas9 and pHERD30T-sgRNA for guide-loaded Cas9 or empty vector for apo-Cas9 were expressed off the PBad promoter, and pMMB67HE-GST-AcrIIA expressed of PLac in P. aeruginosa PAO1 strain. Saturated overnight cultures were diluted 1:100 the next morning in a total volume of 50 mL, induced with 0.3% arabinose and 1 mM IPTG at OD600 0.3-0.4, and harvested at OD600 1.8-2.0 by centrifugation at 6,000×g for 10 mins at 4° C. Cell pellets were flash frozen on dry ice, resuspended in 1 mL lysis buffer (50 mM Tris-Cl pH 7.4, 150 mM NaCl, 20 mM MgCl₂, 0.5% NP40, 5% Glycerol [v/v], 5 mM DTT, and 1 mM PMSF), lysed by sonication (20 s pulse for 4 cycles with cooling on ice between cycles, and lysates were clarified by centrifugation at 14,000×g for 10 mins at 4° C. For input samples, 10 μL lysates were added in 3× volume of 4× Laemmli Sample Buffer. Using a magnetic stand, Anti-c-Myc Magnetic Beads #88842 or Gluthathione Magnetic Agarose Beads #78601 (Thermo Fisher Scientific) were prewashed with 1 mL of cold wash buffer (50 mM Tris-Cl pH 7.4, 150 mM NaCl, 20 mM MgCl₂), and remaining lysate were added to bead slurry in a volume ratio of 20:1 for Myc or 40:1 for GST followed by overnight incubation at 4° C. with end-over-end rotation. Beads were washed five times using a magnetic stand at room temperature with 1 mL of cold wash buffer with addition of 5 mM DTT, gradual decreasing concentrations of detergent NP40 (0.5%, 0.05%, 0.01%, 0.005%, 0) and glycerol (5%, 0.5%, 0.05%, 0.005%, 0). Bead-bound proteins were resuspended in 100 μL of final wash buffer without detergent and glycerol. For analysis, 10 μL of beads-bound protein were added to equal volume of 4× Laemmli Sample Buffer. Samples were analyzed on 4-20% SDS-Page gel and stained with Coomassie (Bio-Safe Coomassie Stain, Bio-Rad).

### Immunoblotting

Protein samples were separated by SDS-Page using 4-20% gel (Mini-PROTEAN TGX Precast Gels, Bio-Rad) and transferred in 1× Tris/Glycine Buffer (Bio-Rad) with 20% Methanol onto 0.2 μm Immun-Blot PVDF Membrane (Bio-Rad). Blots were probed with the following antibodies diluted 1:5000 in 1×TBS-T containing 5% nonfat dry milk: mouse anti-Myc (Cell Signaling Technology #2276, RRID:AB_331783), rabbit anti-GST (Cell Signaling Technology #2625, RRID:AB_490796), mouse anti-E. coli RNA Polymerase 13 (BioLegend #663903, RRID:AB_2564524), HRP-conjugated goat anti-mouse IgG (Santa Cruz Biotechnology #sc-2005, RRID:AB_631736) and HRP-conjugated goat anti-rabbit IgG (Bio-Rad #170-6515, RRID:AB_11125142). Blots were developed using Clarity ECL Western Blotting Substrate (Bio-Rad), and chemiluminescence was detected on an Azure c400 Biosystems Imager.

### Cleavage Assays Using SpyCas9-3xMyc Tagged Pull Downs

DNA substrate linearized by NheI digestion was added into beads-bound protein slurry to a final concentration of 1.5 nM and the reaction was allowed to react for 1, 5, 10 and 30 mins in the thermomixer at 25° C. with gentle shaking 1000 rpm. At each timepoint, the reaction was quenched in warm Quench Buffer (50 mM EDTA, 0.02% SDS), followed by heating at 95° C. for 10 mins. Products were analyzed on 1% agarose gels stained with SYBR Safe.

### REFERENCES

Bhardwaj, P., Ziegler, E., and Palmer, K. L. (2016). Chlorhexidine induces VanA-type vancomycin resistance genes in Enterococci. Antimicrob Agents Chemother 60:2209-2221.
Bondy-Denomy, J., Garcia, B., Strum, S., Du, M., Rollins, M. F., Hidalgo-Reyes, Y., . . . and Davidson, A. R. (2015). Multiple mechanisms for CRISPR-Cas inhibition by anti-CRISPR proteins. Nature, 526(7571), 136-139.
Bondy-Denomy, J., Pawluk, A., Maxwell, K. L., and Davidson, A. R. (2013). Bacteriophage genes that inactivate the CRISPR/Cas bacterial immune system. Nature 493, 429-432.
Borges, A. L., Zhang, J. Y., Rollins, M. F., Osuna, B. A., Wiedenheft, B., and Bondy-Denomy, J. (2018). Bacteriophage Cooperation Suppresses CRISPR-Cas3 and Cas9 Immunity. Cell 174, 917-925.e10.
Bryan, E. M., Bae, T., Kleerebezem, M., and Dunny, G. M. (2000). Improved vectors for nisin-controlled expression in gram-positive bacteria. Plasmid. 44(2): 183-90. 10.
Choi, K.-H., Kumar, A., and Schweizer, H. P. (2006). A 10-min method for preparation of highly electrocompetent Pseudomonas aeruginosa cells: Application for DNA fragment transfer between chromosomes and plasmid transformation. J. Microbiol. Methods 64, 391-397.
Clark, D. P., and Pazdernik, N. J., (2013) Horizontal Gene Transfer. Molecular Evolution. Molecular Biology (Second Edition), e26, e673-679.
Dong, L., Guan, X., Li, N., Zhang, F., Zhu, Y., Ren, K., Yu, L., Zhou, F., Han, Z., Gao, N., et al. (2019). An anti-CRISPR protein disables type V Cas12a by acetylation. Nat. Struct. Mol. Biol. 26, 308-314
Dong, D., Guo, M., Wang, S., Zhu, Y., Wang, S., Xiong, Z., Yang, J., Xu, Z., and Huang, Z. (2017). Structural basis of CRISPR-SpyCas9 inhibition by an anti-CRISPR protein. Nature 546, 436-439
Edgar, R., and Qimron, U. (2010). The Escherichia coli CRISPR system protects from 1 lysogenization, lysogens, and prophage induction. J. Bacteriol. 192, 6291-6294
Forsberg, K. J., Bhatt, I. V., Schmidtke, D. T., Javanmardi, K., Dillard, K. E., Stoddard, B. L., . . . and Malik, H. S. (2019). Functional metagenomics-guided discovery of potent Cas9 inhibitors in the human microbiome. eLife, 8, e46540.
Garneau, J. E., Dupuis, M.-E., Villion, M., Romero, D. A., Barrangou, R., Boyaval, P., Fremaux, C., Horvath, P., Magadan, A. H., and Moineau, S. (2010). The CRISPR/Cas bacterial immune system cleaves bacteriophage and plasmid DNA. Nature, 468, 67-71
Harrington, L. B., Doxzen, K. W., Ma, E., Liu, J.-J., Knott, G. J., Edraki, A., Garcia, B., Amrani, N., Chen, J. S., Cofsky, J. C., et al. (2017). A Broad-Spectrum Inhibitor of CRISPR-Cas9. Cell 170, 1224-1233.e15.
Hoang, T. T., Kutchma, A. J., Becher, A., and Schweizer, H. P. (2000). Integration-Proficient Plasmids for Pseudomonas aeruginosa: Site-Specific Integration and Use for Engineering of Reporter and Expression Strains. Plasmid 43, 59-72.
Hullahalli, K., Rodrigues, M., and Palmer, K. L. (2017). Exploiting CRISPR-Cas to manipulate Enterococcus faecalis populations. eLife, 6, e26664.
Hullahalli, K., Rodrigues, M., Nguyen, U. T., Palmer K. L. (2018). An attenuated CRISPR-Cas system in Enterococcus faecalis permits DNA acquisition. mBio, 9:e00414-18.
Hynes, A. P., Rousseau, G. M., Lemay, M.-L., Horvath, P., Romero, D. A., Fremaux, C., and Moineau, S. (2017). An anti-CRISPR from a virulent streptococcal phage inhibits Streptococcus pyogenes Cas9. Nat. Microbiol. 2, 1374.
Hynes, A. P., Rousseau, G. M., Agudelo, D., Goulet, A., Amigues, B., Loehr, J., . . . and Moineau, S. (2018). Widespread anti-CRISPR proteins in virulent bacteriophages inhibit a range of Cas9 proteins. Nature communications, 9(1), 2919.
Jiang, F., Liu, J.-J., Osuna, B. A., Xu, M., Berry, J. D., Rauch, B. J., Nogales, E., Bondy-Denomy, J., and Doudna, J. A. (2019). Temperature-Responsive Competitive Inhibition of CRISPR-Cas9. Mol. Cell 73, 601-610.e5.
Knott, G. J., Thornton, B. W., Lobba, M. J., Liu, J.-J., Al-Shayeb, B., Watters, K. E., and Doudna, J. A. (2019). Broad-spectrum enzymatic inhibition of CRISPR-Cas12a. Nat. Struct. Mol. Biol. 26, 315-321.
Koonin E. V. (2016). Viruses and mobile elements as drivers of evolutionary transitions. Philosophical transactions of the Royal Society of London. Series B, Biological sciences, 371(1701), 20150442.
Labrie, S. J., Samson, J. E., and Moineau, S. (2010). Bacteriophage resistance mechanisms. Nature reviews. Microbiology, 8, 317-327.
Liu, L., Yin, M., Wang, M., and Wang, Y. (2019). Phage AcrIIA2 DNA Mimicry: Structural Basis of the CRISPR and Anti-CRISPR Arms Race. Mol. Cell, 73, 611-620.e3.
Makarova, K. S., Wolf, Y. I., Alkhnbashi, O. S., Costa, F., Shah, S. A., Saunders, S. J., . . . and Koonin, E. V. (2015). An updated evolutionary classification of CRISPR-Cas systems. Nature reviews. Microbiology, 13(11), 722-736.
Osuna, B. A., Karambelkar, S., Mahendra, C., Christie, K. A., Garcia, B., Davidson, A. R., Kleinstiver, B. P., Kilcher, S., and Bondy-Denomy, J. (2019). Listeria phages protect the genome by triggering Cas9 degradation during lysogeny. bioRxiv. doi.org/10.1101/787200.
Palmer, K. L., and Gilmore, M. S. (2010). Multidrug-resistant enterococci lack CRISPR-cas. mBio, 1(4), e00227-10.
Palmer, K. L., Kos, V. N., and Gilmore, M. S. (2010). Horizontal gene transfer and the genomics of enterococcal antibiotic resistance. Current opinion in microbiology, 13(5), 632-639.
Pawluk, A., Amrani, N., Zhang, Y., Garcia, B., Hidalgo-Reyes, Y., Lee, J., Edraki, A., Shah, M., Sontheimer, E. J., Maxwell, K. L., et al. (2016). Naturally Occurring Off-Switches for CRISPR-Cas9. Cell 167, 1829-1838.e1829.
Price, V. J., Huo, W., Sharifi, A., and Palmer, K. L. (2016). CRISPR-Cas and Restriction-Modification Act Additively against Conjugative Antibiotic Resistance Plasmid Transfer in Enterococcus faecalis. mSphere, 1(3), e00064-16.
Rauch, B. J., Silvis, M. R., Hultquist, J. F., Waters, C. S., McGregor, M. J., Krogan, N. J., and Bondy-Denomy, J. (2017). Inhibition of CRISPR-Cas9 with Bacteriophage Proteins. Cell, 168(1-2), 150-158.e10.
Seamon, K. J., Light, Y. K., Saada, E. A., Schoeniger, J. S., and Harmon, B. (2018). Versatile High-Throughput Fluorescence Assay for Monitoring Cas9 Activity. Analytical Chemistry, 90 (11), 6913-6921.
Shin, J., Jiang, F., Liu, J. J., Bray, N. L., Rauch, B. J., Baik, S. H., . . . and Doudna, J. A. (2017). Disabling Cas9 by an anti-CRISPR DNA mimic. Science advances, 3(7), e1701620.
Trasanidou, D., GerOs, A. S., Mohanraju, P., Nieuwenweg, A. C., Nobrega, F. L., and Staals, R. (2019). Keeping crispr in check: diverse mechanisms of phage-encoded anti-crisprs. FEMS microbiology letters, 366(9), fnz098.
Uribe, R. V., van der Helm, E., Misiakou, M.-A., Lee, S.-W., Kol, S., and Sommer, M. O. A. (2019). Discovery and Characterization of Cas9 Inhibitors Disseminated across Seven Bacterial Phyla. Cell Host & Microbe, 25(2): 233-241.e5.
Waldor, M. K., and Mekalanos, J. J. (1996). Lysogenic Conversion by a Filamentous Phage Encoding Cholera Toxin. Science, 272(5270): 1910-1914.
Zhang, Y., Heidrich, N., Ampattu, B. J., Gunderson, C. W., Seifert, H. S., Schoen, C., . . . and Sontheimer, E. J. (2013). Processing-independent CRISPR RNAs limit natural transformation in Neisseria meningitidis. Molecular Cell, 50(4), 488-503.
Zhang, F., Song, G., and Tian, Y. (2019). Anti-CRISPRs: The natural inhibitors for CRISPR-Cas systems. Animal models and experimental medicine, 2(2), 69-75.
Zhu, Y., Gao, A., Zhan, Q., Wang, Y., Feng, H., Liu, S., Gao, G., Serganov, A., and Gao, P. (2019). Diverse Mechanisms of CRISPR-Cas9 Inhibition by Type IIC Anti-CRISPR Proteins. Mol. Cell 74, 296-309.e7.

TABLE 2A
Bacterial strains used in the study

| Name | Bug | Strain | Genotype | Plasmid | Plasmid Link | Resistance |
|---|---|---|---|---|---|---|
| bCM018 | P. aeruginosa | PAO1 | tn7pLac::spyCas9 | pUC18-Tn7-lac | | Gent 50 |
| bCM022 | E. coli | DH5a | pUC-pLac::spyCas9 | pUC18-Tn7- | | Gent 30 |
| | | | | Lac::spyCas9 | | |
| bCM037 | E. coli | DH5a | mini-CTX2-pLac | pCM5 | benchling.com/s/seq- | Tet 10 |
| | | | | | pcO0qZtbMvAln67bq7hT | |
| bCM038 | E. coli | DH5a | AcrIIA4 | pCM6 | benchling.com/s/seq- | Gent 30 |
| | | | | | IQdlb5g575NZufBXnRVa | |
| bCM039 | P. aeruginosa | PAO1 | tn7pLac::spyCas9 | pUC18-Tn7-lac | | |
| bCM040 | E. coli | DH5a | ctxpLac::sgRNA-Bsalsites | pCM7 | benchling.com/s/seq- | Tet 10 |
| | | | | | hBDIwnJjRKiliQbKZT67 | |
| bCM041 | E. coli | DH5a | ctx2pLac::sgRNA-JBD30 | pCM8 | benchling.com/s/seq- | Tet 10 |
| | | | | | MxNZDuWiktc0YjbWfy20 | |
| bCM045 | P. aeruginosa | PAO1 | tn7pLac::spyCas0, | | | |
| | | | ctx2pLac::sgRNA-Bsalsites | | | |
| bCM046 | P. aeruginosa | PAO1 | tn7pLac::spyCas0, | | | |
| | | | ctx2pLac::sgRNA-JBD30 | | | |
| bCM047 | P. aeruginosa | PAO1 | tn7pLac::spyCas9, | pMMB67HE ev | | Carb 250 |
| | | | pMMB67HE-EV | | | |
| bCM048 | P. aeruginosa | PAO1 | tn7pLac::spyCas9, | pMMB67HE::s | benchling.com/s/seq- | Carb 250 |
| | | | pMMB67HE-sgJBD30 | gCas9_JBD30 | QmcCNgobGcvdfaHjOKpM | |
| bCM049 | P. aeruginosa | PAO1 | tn7pLac::spyCas9 , | pMMB67HE::e | | Gent 30, |
| | | | pMMB67HE-EV, | v, | | Carb 250 |
| | | | pHERD30T-EV | pHERD30T::ev | | |
| bCM051 | P. aeruginosa | PAO1 | tn7pLac::spyCas9, | pMMB67HE::s | | Gent 30, |
| | | | pMMB67HE::sgJBD30, | gCas9_JBD30, | | Carb 250 |
| | | | pHERD30T-EV | pHERD30T::ev | | |
| bCM052 | P. aeruginosa | PAO1 | tn7pLac::spyCas9, | pMMB67HE::s | | Gent 30, |
| | | | pMMB67HE::sgJBD30, | gCas9_JBD30, | | Carb 250 |
| | | | AcrIIA4 | pCM6 | | |
| bCM053 | P. aeruginosa | PAO1 | tn7pLac::spyCas9, | pHERD30T::ev | | Gent 50 |
| | | | ctx2pLac::sgBsal, | | | |
| | | | pHERD30T-EV | | | |
| bCM055 | P. aeruginosa | PAO1 | tn7pLac::spyCas9, | pHERD30T::ev | | Gent 50 |
| | | | ctx2pLac::sgJBD30, | | | |
| | | | pHERD30T-EV | | | |
| bCM056 | P. aeruginosa | PAO1 | tn7pLac::spyCas9, | pCM6 | | Gent 50 |
| | | | ctx2pLac::sgJBD30, AcrIIA4 | | | |
| bCM068 | P. aeruginosa | PAO1 | tn7pLac::dSpyCas9 | | | |
| bCM079 | E. coli | DH5a | AcrIIA16_Lmo | pCM28 | benchling.com/s/seq- | Gent 30 |
| | | | | | buYB9nI2BRCcxsF1a9S0 | |
| bCM085 | E. coli | DH5a | sgRNA-PhzM5 | pCM11 | benchling.com/s/seq- | Tet 10 |
| | | | | | 35qjyzqBYltrtNN23kiP | |
| bCM100 | P. aeruginosa | PAO1 | tn7pLac::spyCas9, | pCM28 | | Gent 50 |
| | | | ctx2pLac::sgJBD30, | | | |
| | | | AcrIIA16_Lmo | | | |
| bCM153 | E. coli | DH5a | AcrIIA16_Lmo | pCM38 | benchling.com/s/seq- | Carb 100 |
| | | | | | sCfcUF2qBXtWQvZ6y2II | |
| bCM154 | P. aeruginosa | PAO1 | tn7pLac::spyCas9, | pMMB67HE::s | | Gent 30, |
| | | | pMMB67HE::sgJBD30, | gJBD30, | | Carb 100 |
| | | | AcrIIA16_Lmo | pCM28 | | |
| bCM155 | E. coli | Turbo | AcrIIA17_Efa | pCM45 | benchling.com/s/seq- | Gent 30 |
| | | | | | 1mMtrBikmxsTt3mKTO8g | |
| bCM156 | E. coli | Turbo | AcrIIA17_Sga | pCM46 | benchling.com/s/seq- | Gent 30 |
| | | | | | be1xgfFVSFxPgHogpOmd | |
| bCM159 | E. coli | Turbo | AcrIIA16_Lmo | pCM48 | benchling.com/s/seq- | Kan 25 |
| | | | | | WvAhUZYIIPB9dvVf3Vst | |
| bCM160 | E. coli | Turbo | AcrIIA18_Sma | pCM49 | benchling.com/s/seq- | Gent 30 |
| | | | | | SndExMkJ6QkLUqiltibE | |
| bCM173 | P. aeruginosa | PAO1 | tn7pLac::spyCas9, | pMMB67HE::s | | Gent 30, |
| | | | pMMB67HE::sgJBD30, | gJBD30, | | Carb 100 |
| | | | AcrIIA17_Efa | pCM45 | | |
| bCM174 | P. aeruginosa | PAO1 | tn7pLac::spyCas9, | pMMB67HE::s | | Gent 30, |
| | | | pMMB67HE::sgJBD30, | gJBD30, | | Carb 100 |
| | | | AcrIIA17_Sga | pCM46 | | |
| bCM176 | P. aeruginosa | PAO1 | tn7pLac::spyCas9, | pMMB67HE::s | | Gent 30, |
| | | | pMMB67HE::sgJBD30, | gJBD30, | | Carb 100 |
| | | | AcrIIA18_Sma | pCM49 | | |
| bCM178 | P. aeruginosa | PAO1 | tn7pLac::spyCas9, | pCM45 | | Gent 50 |
| | | | ctx2pLac::sgJBD30, | | | |
| | | | AcrIIA17_Efa | | | |
| bCM180 | P. aeruginosa | PAO1 | tn7pLac::spyCas9, | pCM49 | | Gent 50 |
| | | | ctx2pLac::sgJBD30, | | | |
| | | | AcrIIA18_Sma | | | |
| bCM196 | P. aeruginosa | PAO1 | tn7pLac::spyCas9, | pCM46 | | Gent 50 |
| | | | ctx2pLac::sgJBD30, | | | |
| | | | AcrIIA17_Sga | | | |
| bCM198 | P. aeruginosa | PAO1 | tn7pBAD::spyCas9, | 30T::sgJBD30, | | Gent 30, |
| | | | 30T::sgJBD30, | pMMB67HE::e | | Carb 100 |
| | | | pMMB67HE::ev | v | | |
| bCM200 | P. aeruginosa | PAO1 | tn7pBAD::spyCas9, | 30T::sgJBD30, | | |
| | | | 30T::sgJBD30, AcrIIA4 | pMMB67HE::A | | |
| | | | | crIIA4 | | |
| bCM202 | P. aeruginosa | PAO1 | tn7pBAD::spyCas9, | pHERD30T::sg | | Gent 50 |
| | | | 30T::sgJBD30 | JBD30 | | |
| bCM235 | E. coli | DH5a | AcrIIA18_Sma | pCM78 | benchling.com/s/seq- | Kan 25 |
| | | | | | SHYFRiGAcmTRiXyOxsnQ | |
| bCM236 | E. coli | BL21 | AcrIIA17_Sga | pCM70 | | Kan 25 |
| bCM239 | E. coli | BL21 | AcrIIA16_Lmo | pCM48 | | Kan 25 |
| bCM246 | E. coli | BL21 | AcrIIA18_Sma | pCM78 | | Kan 25 |
| bCM248 | E. coli | EC1000 | pKH12 | | | Chlo 25 |
| bCM249 | E. faecalis | OG1RF | wt | | | Rif 50 |
| bCM250 | E. faecalis | T11RF | wt | | | Rif 50 |
| bCM251 | E. faecalis | T11RF | Δcas9 | | | Rif 50 |
| bCM252 | E. faecalis | C173 | wt | | | Spec 500, |
| | | | | | | Strep 500 |
| bCM262 | P. aeruginosa | PAO1 | tn7pLac::spyCas9, | pCM110 | | Carb 250 |
| | | | pMMB67HE::sgPhzm5 | | | |
| bCM263 | P. aeruginosa | PAO1 | tn7pLac::dCas9, | pCM110 | | Carb 250 |
| | | | pMMB67HE::sgPhzm5 | | | |
| bCM271 | E. faecalis | C173 | pKH12 | pKH12 | | Spec 500, |
| | | | | | | Strep 500, |
| | | | | | | Chlo 15 |
| bCM272 | E. coli | | pMSP3535 | pCM116 | | Erm 300 |
| bCM273 | E. faecalis | C173 | pKH12::CR3S6 | pCM118 | benchling.com/s/seq- | Spec 500, |
| | | | | | wj3NsaNMGeaj0jVTCNYa | Strep 500, |
| | | | | | | Chlo 15 |
| bCM283 | E. coli | Inoue | pKH CR1S96 | pCM117 | benchling.com/s/seq- | Chlo 25 |
| | | | | | IplfloODeopBvE3xxuFZ | |
| bCM284 | E. coli | Inoue | pKH CR3S6 | pCM118 | benchling.com/s/seq- | Chlo 25 |
| | | | | | wj3NsaNMGeaj0jVTCNYa | |
| bCM285 | E. faecalis | C173 | pKH12::CR1S96 | pCM117 | benchling.com/s/seq- | Spec 500, |
| | | | | | IplfloODeopBvE3xxuFZ | Strep 500, |
| | | | | | | Chlo 15 |
| bCM313 | E. faecalis | OG1RF | EV | pCM116 | | Rif, Fus, |
| | | | | | | Erm 50 |
| bCM319 | E. faecalis | T11RF | EV | pCM116 | | Rif, Fus, |
| | | | | | | Erm 50 |
| bCM320 | E. faecalis | OG1RF | Efae promoter, | pCM125 | | Rif, Fus, |
| | | | AcrIIA16_Lmo | | | Erm 50 |
| bCM321 | E. faecalis | T11RF | Efae promoter, | pCM125 | | Rif, Fus, |
| | | | AcrIIA16_Lmo | | | Erm 50 |
| bCM322 | E. faecalis | OG1RF | Efae promoter, AcrIIA4 | pCM126 | | Rif, Fus, |
| | | | | | | Erm 50 |
| bCM323 | E. faecalis | OG1RF | Efae promoter, | pCM127 | | Rif, Fus, |
| | | | AcrIIA17_Efa | | | Erm 50 |
| bCM324 | E. faecalis | OG1RF | Efae promoter, | pCM128 | | Rif, Fus, |
| | | | AcrIIA17_Sga | | | Erm 50 |
| bCM325 | E. faecalis | OG1RF | Efae promoter, | pCM129 | | Rif, Fus, |
| | | | AcrIIA19_Ssim | | | Erm 50 |
| bCM328 | E. faecalis | T11RF | Efae promoter, AcrIIA4 | pCM126 | | Rif, Fus, |
| | | | | | | Erm 50 |
| bCM329 | E. faecalis | T11RF | Efae promoter, | pCM127 | | Rif, Fus, |
| | | | AcrIIA17_Efa | | | Erm 50 |
| bCM330 | E. faecalis | T11RF | Efae promoter, | pCM128 | | Rif, Fus, |
| | | | AcrIIA17_Sga | | | Erm 50 |
| bCM331 | E. faecalis | T11RF | Efae promoter, | pCM129 | | Rif, Fus, |
| | | | AcrIIA19_Ssim | | | Erm 50 |
| bCM337 | E. faecalis | C173 | pKH-CR1 :: Efae promoter, | pCM135 | | Strep, |
| | | | AcrIIA16_Lmo | | | Spec 50, |
| | | | | | | Chlo 15 |
| bCM338 | E. faecalis | C173 | pKH-CR1 :: Efae promoter, | pCM137 | | Strep, |
| | | | AcrIIA17_Efa | | | Spec 50, |
| | | | | | | Chlo 15 |
| bCM339 | E. faecalis | C173 | pKH-CR1 :: Efae promoter, | pCM138 | | Strep, |
| | | | AcrIIA17_Sga | | | Spec 50, |
| | | | | | | Chlo 15 |
| bCM340 | E. faecalis | C173 | pKH-CR1 :: Efae promoter, | pCM139 | | Strep, |
| | | | AcrIIA19_Ssim | | | Spec 50, |
| | | | | | | Chlo 15 |
| bCM341 | E. faecalis | C173 | pKH-CR3 :: Efae promoter, | pCM142 | | Strep, |
| | | | AcrIIA16_Lmo | | | Spec 50, |
| | | | | | | Chlo 15 |
| bCM342 | E. faecalis | C173 | pKH-CR3 :: Efae promoter, | pCM143 | | Strep, |
| | | | AcrIIA4 | | | Spec 50, |
| | | | | | | Chlo 15 |
| bCM343 | E. faecalis | C173 | pKH-CR3 :: Efae promoter, | pCM144 | | Strep, |
| | | | AcrIIA17_Efa | | | Spec 50, |
| | | | | | | Chlo 15 |
| bCM344 | E. faecalis | C173 | pKH-CR3 :: Efae promoter, | pCM145 | | Strep, |
| | | | AcrIIA17_Sga | | | Spec 50, |
| | | | | | | Chlo 15 |
| bCM347 | E. coli | BL21 | AcrIIA19_Ssim | pCM132 | | Kan 25 |
| bCM348 | E. coli | XL1-blue | VEGFA2 protospacer | pCM133 | | Gent 10 |
| bCM350 | E. faecalis | C173 | pKH-CR1 :: Efae promoter, | pCM136 | | Strep, |
| | | | AcrIIA4 | | | Spec 50, |
| | | | | | | Chlo 15 |
| bCM352 | E. faecalis | C173 | pKH-CR3 :: Efae promoter, | pCM146 | | Strep, |
| | | | AcrIIA19_Ssim | | | Spec 50, |
| | | | | | | Chlo 15 |
| bCM353 | E. coli | Turbo | JBD30 IIA protospacer | pCM149 | benchling.com/s/seq- | Gent 10 |
| | | | | | rEeZCqLFhUskCR0FBvwm | |
| bCM358 | P. aeruginosa | PAO1 | tn7pBAD::spyCas9, | 30T-gJBD30, | | Gent30, |
| | | | sgJBD30, GST-AcrIIA4 | pMMB67HE::G | | Carb100 |
| | | | | ST-AcrIIA4 | | |
| bCM359 | P. aeruginosa | PAO1 | tn7pBAD::spyCas9, | 30T-gJBD30, | | Gent30, |
| | | | sgJBD30, GST-AcrIIA2b.3 | pMMB67HE::G | | Carb100 |
| | | | | ST-AcrIIA2b.3 | | |
| bCM361 | P. aeruginosa | PAO1 | tn7pBAD::spyCas9, | 30T-gJBD30, | benchling.com/s/seq- | Gent30, |
| | | | sgJBD30, GST- | pCM150 | Rlu4DGKrPLZ02ASg6eD5 | Carb100 |
| | | | AcrIIA16_Lmo | | | |
| bCM362 | P. aeruginosa | PAO1 | tn7pBAD::spyCas9, | 30T-gJBD30, | benchling.com/s/seq- | Gent30, |
| | | | sgJBD30, GST-AcrIIA7_Sga | pCM151 | GN6tS6v9oPKA4OJ1r4g9 | Carb100 |
| bCM363 | P. aeruginosa | PAO1 | tn7pBAD::spyCas9, | 30T-gJBD30, | benchling.com/s/seq- | Gent30, |
| | | | sgJBD30, GST- | pCM152 | T3XKs8xXQ0oMElj27XDZ | Carb100 |
| | | | AcrIIA18_Sma | | | |
| bCM364 | P. aeruginosa | PAO1 | tn7pBAD::spyCas9, | 30T-gJBD30, | benchling.com/s/seq- | Gent30, |
| | | | sgJBD30, GST- | pCM153 | qu45PDP2tf9rPGkVPLCh | Carb100 |
| | | | AcrIIA19_Ssim | | | |
| bCM365 | P. aeruginosa | PAO1 | tn7pBAD::spyCas9, | pHERD30T-EV | | Gent50 |
| | | | pHERD30T-EV | | | |
| bCM366 | P. aeruginosa | PAO1 | tn7pBAD::spyCas9, | 30T-gJBD30, | | Gent30, |
| | | | sgJBD30, GST-EV | pMMB67HE::G | | Carb100 |
| | | | | ST-EV | | |
| bCMP3- | P. aeruginosa | PAO1 | tn7pLac::SpCas9, | pHERD30T-EV | | Gent30, |
| A1 | | | pMMB67HE-EV, | | | Carb100 |
| | | | pHERD30T-EV | | | |
| bCMP3- | P. aeruginosa | PAO1 | tn7pLac::SpCas9, | pHERD30T-EV | | Gent30, |
| A2 | | | pMMB67HE-sgJBD30, | | | Carb100 |
| | | | pHERD30T-EV | | | |
| bCMP3- | P. aeruginosa | PAO1 | tn7pLac::SpCas9, | pCM6 | | Gent30, |
| A3 | | | pMMB67HE-sgJBD30, | | | Carb100 |
| | | | pHERD30T-AcrIIA4 | | | |
| bCMP3- | P. aeruginosa | PAO1 | tn7pLac::SpCas9, | pCM28 | | Gent30, |
| A4 | | | pMMB67HE-sgJBD30, | | | Carb100 |
| | | | pHERD30T-AcrIIA16_Lmo | | | |
| bCMP3- | P. aeruginosa | PAO1 | tn7pLac::SpCas9, | pCM45 | | Gent30, |
| A5 | | | pMMB67HE-sgJBD30, | | | Carb100 |
| | | | pHERD30T-AcrIIA17_Efa | | | |
| bCMP3- | P. aeruginosa | PAO1 | tn7pLac::SpCas9, | pCM46 | | Gent30, |
| A6 | | | pMMB67HE-sgJBD30, | | | Carb100 |
| | | | pHERD30T-AcrIIA17_Sga | | | |
| bCMP3- | P. aeruginosa | PAO1 | tn7pLac::SpCas9, | pCM49 | | Gent30, |
| A7 | | | pMMB67HE-sgJBD30, | | | Carb100 |
| | | | pHERD30T-AcrIIA18_Sma | | | |
| bCMP3- | P. aeruginosa | PAO1 | tn7pLac::SpCas9, | pCM83 | | Gent30, |
| A8 | | | pMMB67HE-sgJBD30, | | | Carb100 |
| | | | pHERD30T-AcrIIA19_Ssim | | | |
| bCMP3- | P. aeruginosa | PAO1 | tn7pLac::SpCas9, | pHERD30T-EV | | Gent30, |
| C2 | | | pMMB67HE-sgPhzM5, | | | Carb100 |
| | | | pHERD30T-EV | | | |
| bCMP3- | P. aeruginosa | PAO1 | tn7pLac::SpCas9, | pCM6 | | Gent30, |
| C3 | | | pMMB67HE-sgPhzM5, | | | Carb100 |
| | | | pHERD30T-AcrIIA4 | | | |
| bCMP3- | P. aeruginosa | PAO1 | tn7pLac::SpCas9, | pCM28 | | Gent30, |
| C4 | | | pMMB67HE-sgPhzM5, | | | Carb100 |
| | | | pHERD30T-AcrIIA16_Lmo | | | |
| bCMP3- | P. aeruginosa | PAO1 | tn7pLac::SpCas9, | pCM45 | | Gent30, |
| C5 | | | pMMB67HE-sgPhzM5, | | | Carb100 |
| | | | pHERD30T-AcrIIA17_Efa | | | |
| bCMP3- | P. aeruginosa | PAO1 | tn7pLac::SpCas9, | pCM46 | | Gent30, |
| C6 | | | pMMB67HE-sgPhzM5, | | | Carb100 |
| | | | pHERD30T-AcrIIA17_Sga | | | |
| bCMP3- | P. aeruginosa | PAO1 | tn7pLac::SpCas9, | pCM49 | | Gent30, |
| C7 | | | pMMB67HE-sgPhzM5, | | | Carb100 |
| | | | pHERD30T-AcrIIA18_Sma | | | |
| bCMP3- | P. aeruginosa | PAO1 | tn7pLac::SpCas9, | pCM83 | | Gent30, |
| C8 | | | pMMB67HE-sgPhzM5, | | | Carb100 |
| | | | pHERD30T-AcrIIA19_Ssim | | | |
| bCMP3- | P. aeruginosa | PAO1 | tn7pLac::dCas9, | pHERD30T-EV | | Gent30, |
| E2 | | | pMMB67HE-sgPhzM5, | | | Carb100 |
| | | | pHERD30T-EV | | | |
| bCMP3- | P. aeruginosa | PAO1 | tn7pLac::dCas9, | pCM6 | | Gent30, |
| E3 | | | pMMB67HE-sgPhzM5, | | | Carb100 |
| | | | pHERD30T-AcrIIA4 | | | |
| bCMP3- | P. aeruginosa | PAO1 | tn7pLac::dCas9, | pCM28 | | Gent30, |
| E4 | | | pMMB67HE-sgPhzM5, | | | Carb100 |
| | | | pHERD30T-AcrIIA16_Lmo | | | |
| bCMP3- | P. aeruginosa | PAO1 | tn7pLac::dCas9, | pCM45 | | Gent30, |
| E5 | | | pMMB67HE-sgPhzM5, | | | Carb100 |
| | | | pHERD30T-AcrIIA17_Efa | | | |
| bCMP3- | P. aeruginosa | PAO1 | tn7pLac::dCas9, | pCM46 | | Gent30, |
| E6 | | | pMMB67HE-sgPhzM5, | | | Carb100 |
| | | | pHERD30T-AcrIIA17_Sga | | | |
| bCMP3- | P. aeruginosa | PAO1 | tn7pLac::dCas9, | pCM49 | | Gent30, |
| E7 | | | pMMB67HE-sgPhzM5, | | | Carb100 |
| | | | pHERD30T-AcrIIA18_Sma | | | |
| bCMP3- | P. aeruginosa | PAO1 | tn7pLac::dCas9, | pCM83 | | Gent30, |
| E8 | | | pMMB67HE-sgPhzM5, | | | Carb100 |
| | | | pHERD30T-AcrIIA19_Ssim | | | |

TABLE 2B
Plasmids used in the study

| Name | Background | Genotype | Plasmid Link |
|---|---|---|---|
| pCM005 | CTX2 | mini-CTX2-lac | benchling.com/s/seq-pcOOqZtbMvAln67bq7hT |
| pCM006 | PHERD30T | AcrIIA4 | benchling.com/s/seq-IQdlb5g575NZufBXnRVa |
| pCM007 | pCM5 | sg RNA-SpCas9-Bsal | benchling.com/s/seq-hBDIwnJjRKiliQbKZT67 |
| pCM008 | pCM5 | sgRNA-SpCas9-JBD30 | benchling.com/s/seq-MxNZDuWiktc0YjbWfy20 |
| pCMO11 | pCM7 | sgRNA-SpCas9-PhzM5 | benchling.com/s/seq-35qjyzqBYItrtNN23kiP |
| pCM028 | PHERD30T | AcrIIA16_Lmo | benchling.com/s/seq-buYB9nl2BRCcxsF1a9S0 |
| pCM038 | PMMB67HE | AcrIIA16_Lmo | benchling.com/s/seq-sCfcUF2qBXtWQvZ6y2ll |
| pCM045 | PHERD30T | AcrIIA17_Efa | benchling.com/s/seq-1mMtrBikmxsTt3mKTO8g |
| PCM046 | PHERD30T | AcrIIA17_Sga | benchling.com/s/seq-be1xgfFVSFxPgHogpOmd |
| PCM048 | pET28 | AcrIIA16_Lmo | benchling.com/s/seq-WvAhUZYIIPB9dvVf3Vst |
| PCM049 | pHERD30T | AcrIIA18_Sma | benchling.com/s/seq-SndExMkJ6QkLUqiltibE |
| pCM070 | PET28 | AcrIIA17_Sga | benchling.com/s/seq-skz7bOaQ4KAP7Tr3REL5 |
| pCM078 | PET28 | AcrIIA18_Sma | benchling.com/s/seq-SHYFRiGAcmTRiXyOxsnQ |
| PCM083 | PHERD30T | AcrIIA19_Ssim | benchling.com/s/seq-OCDuuwHPzkXZN5aldGNo |
| PCM096 | pKH12 | PKH12-EV | benchling.com/s/seq-XFN4NQULYef2kGAJ7yyx |
| PCM116 | pMSP3535 | pMSP3535-EV | benehling.eom/s/seq-we9nrxP5AGXwMrjpGLh7 |
| pCM117 | pKH12 | CR1-S96 | benchling.com/s/seq-lplfloODeopBvE3xxuFZ |
| pCM118 | pKH12 | CR3-S6 | benchling.com/s/seq-wj3NsaNMGeaj0iVTCNYa |
| pCM125 | pMSP3535 | Efae promoter, AcrIIA16_Lmo | benchling.com/s/seq-d KLI447GcniB235u7LK |
| PCM126 | pMSP3535 | Efae promoter, AcrIIA4 | benchling.com/s/seq-hJRjaEPXXvflABZBIuB2 |
| pCM127 | pMSP3535 | Efae promoter, AcrIIA17_Efa | benchling.com/s/seq-oBRwnqeuPpxdma8YN4mp |
| pCM128 | pMSP3535 | Efae promoter, AcrIIA17_Sga | benchling.com/s/seq-plp8eWnTCaH76ZoellBB |
| pCM129 | pMSP3535 | Efae promoter, AcrIIA19_Ssim | benchling.com/s/seq-rp9e2mHHIfnQQAQTIE1S |
| pCM132 | pET28 | AcrIIAI 9 Ssim | benehling.eom/s/seq-HzkjlqsohGNkRABI3PWS |
| pCM133 | PHERD30T | VEGFA protospacer | benchling.com/s/seq-Xzlp1bxKBqjD8leQ9JFa |
| pCM135 | pCM117 | CR1, Efae promoter, | benchling.com/s/seq-wWWMmOII5Xeph364ZrE8 |
| | | AcrIIA16_Lmo | |
| pCM136 | pCM117 | CR1, Efae promoter, AcrIIA4 | benchling.com/s/seq-pe8libQxvpYkPwDfuwgH |
| pCM137 | pCM117 | CR1, Efae promoter, | benchling.com/s/seq-pqGFn4SjC1D3KASJhCDy |
| | | AcrIIAI 7 Efa | |
| pCM138 | pCM117 | CR1, Efae promoter, | benchling.com/s/seq-DcGbk3dNnUhxvPK5q428 |
| | | AcrIIAI 7 Sga | |
| pCM139 | pCM117 | CR1, Efae promoter, | benchling.com/s/seq-qshXPjlcxj7u9NVbWqL2 |
| | | AcrIIAI 9 Ssim | |
| pCM142 | pCM118 | CR3, Efae promoter, | benchling.com/s/seq-5sPKYJ8SAccqqjWRLvEi |
| | | AcrIIAI 6 Lmo | |
| pCM143 | pCM118 | CR3, Efae promoter, AcrIIA4 | benchling.com/s/seq-xDqEBSG98omFjlAzhsSh |
| pCM144 | pCM118 | CR3, Efae promoter, | benchling.com/s/seq-bTzsIUxZXAOkckfJOdqb |
| | | AcrIIA17_Efa | |
| pCM145 | pCM118 | CR3, Efae promoter, | benchling.com/s/seq-an430GQD9XSgMfVyd0Gj |
| | | AcrIIA17_Sga | |
| pCM146 | pCM118 | CR3, Efae promoter, | benchling.com/s/seq-oXTWcnWk46PW0mQlaZjr |
| | | AcrIIA19_Ssim | |
| pCM149 | pHERD30T | JBD30 IIA protospacer | benchling.com/s/seq-rEeZCqLFhUskCROFBvwm |
| pCM150 | PMMB67HE | GST-AcrIIA16 Lmo | benchling.com/s/seq-Rlu4DGKrPLZ02ASg6eD5 |
| pCM151 | PMMB67HE | GST-AcrIIA17_Sga | benchling.com/s/seq-GN6tS6v9oPKA4OJ1r4g9 |
| PCM152 | PMMB67HE | GST-AcrIIA18_Sma | benchling.com/s/seq-T3XKs8xXQ0oMEIj27XDZ |
| pCM153 | PMMB67HE | GST-AcrIIA19_Ssim | benchling.com/s/seq-qu45PDP2tf9rPGkVPLCh |
| pCM005 | CTX2 | mini-CTX2-lac | benchling.com/s/seq-pcOOqZtbMvAln67bq7hT |
| PCM006 | PHERD30T | AcrIIA4 | benchling.com/s/seq-IQdlb5g575NZufBXnRVa |
| pCM007 | pCM5 | sgRNA-SpCas9-Bsal | benchling.com/s/seq-hBDIwnJjRKiliQbKZT67 |
| PCM008 | pCM5 | sgRNA-SpCas9-J BD30 | benchling.com/s/seq-MxNZDuWiktc0YjbWfy20 |
| pCM011 | pCM7 | sgRNA-SpCas9-PhzM5 | benehling.eom/s/seq-35qjyzqBYItrtNN23kiP |
| PCM028 | pHERD30T | AcrllA16 Lmo | benchling.com/s/seq-buYB9nl2BRCcxsF1a9S0 |
| PCM038 | PMMB67HE | AcrllA16 Lmo | benchling.com/s/seq-sCfcUF2qBXtWQvZ6y2ll |
| PCM045 | PHERD30T | AcrllA17 Efa | benchling.com/s/seq-1mMtrBikmxsTt3mKTO8g |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

INFORMAL SEQUENCE LISTING
AcrIIA16 protein sequences
IIA16-Lmo
Listeria monocytogenes
SEQ ID NO: 1
MGYIGTKRSERSQDAIEDYEVPLNHFNKDLIQAFIDENEAYDTLKTKKV
RLWKFVAPRAGATSWHHTGTYYNKTDHYSLEKVADELLQNGDEWEEQFK
AYVKEEQETATSEPVFLSVIKVQIWGGSMKRPKLVGHEVVMGVKKEGWL
HAVSKATQSKYKLSANKVEMQKHYSLEDYSALTKDFPEFKAQKRAINKK
MKEMYN
IIA16-Efa
Enterococcus faecalis
SEQ ID NO: 2
MGYVGKSRSVRSQIAIDNAEVPLNHITKDYILTFVTENNIDETLKNESV
AMWKFVAKRHGSTSWHHVSKHYNKIDHYDLHDVAEYFSMNYDSLKNDYQ
NLLDQKRQAKNDLIKNLKLGIIKVQIWGGTKRYPKLEGYESVMGVVKDG
WLHTVTLSNQTKYKITGNKIEEITIFELDQYDILTKKFPEFRAMKRKIN
KEVARLSK
AcrIIA17 protein sequences
IIA17-Efa
Enterococcus faecalis
SEQ ID NO: 3
MAILNNKGEKISIDCADLISEVEEDILIFGGTFLVYAICSWREIEQVEY
ISDYVHADNPESYKDELTTKEYAELKEIYEKDLEELKITKNKQMNLNEL
LSILTIQNSIT
IIA17-Sga
Streptococcus gallolyticus
SEQ ID NO: 4
MKISVDSEKLLNEAINDFDIFGEDFNVYAIYSYREDYDFEYISDYVDAD
EPTRDEFETEEDYQEVMKDFKENLDSLKFTKHKKMTIADLVHELWEQNR
IF
AcrIIA18 protein sequences
IIA18-Sma
Streptococcus macedonicus
SEQ ID NO: 5
MKIDTTVTEVKENGKTYLRLLKGNEQLKAVSDKAVAGVNLFPGAKIGSF
LVRQDNIVVFPDNKGEFDLDFFNLLNDNFETLVEYAKMADCLDIAFDIN
EKSYFNMIMWLMKNIDENWSQSPYGESFYSSKDIDWGYKPEGSLRVSDH
WNFGQDGEHCPTAEPVDGWAVCKFENGKYHLIKKF
IIA18-Sga
Streptococcus gallolyticus
SEQ ID NO: 6
MKIDTTVTEVKENGKTYLRLVEGTEQLKAISDKAMAGVNLFPGAKIDSF
LVKQDSIVVFPDNKGEFDLDFFKQLDENFDTIAKYARVATCFEEVAFDE
KSYFNMIMWLMDNMDENWSQSPYGESFYSSKNIDWGYKPEGSLRVSDHW
NFGENGEHCPTAEPVDGWAVCKFENGKYHLIKKF
AcrIIA19 protein sequences
IIA19-Ssim
Staphylococcus simulans
SEQ ID NO: 7
MKLIVEVEETNYKNLVNYTKLTNESHNILVNRLISEYITKPYELRLDLS
ERYSNRDLIEFKFMLIEYCKEALQDIKELANSDEAYETDEAFEAVFRQL
FEEVISNPDTVLKAFHSYTSFLEENK
IIA19-Spseu
Staphylococcus pseudintermedius
SEQ ID NO: 8
MKLIINIEDKNYKYLTELAQQDNTNIGSIVNNLIQTHITDVNESYRSVD
KKELDEFSRVMQHYFHEDLASMYDVIGSDEELSTDKQMLKVYKKLYQDV
ALRNGIALELFNAYKKG

1. A method of inhibiting a Cas9 polypeptide in a cell, the method comprising,
introducing a Cas9-inhibiting polypeptide into a cell, wherein:
the Cas9-inhibiting polypeptide is heterologous to the cell, and
the Cas9-inhibiting polypeptide is at least 95% identical to any one or more of SEQ ID NOS: 1-8;
thereby inhibiting the Cas9 polypeptide in the cell.

2. (canceled)

3. The method of claim 1, wherein the Cas9-inhibiting polypeptide comprises the amino acid sequence of any one of SEQ ID NOS: 1-8.

4. (canceled)

5. The method of claim 1, wherein the cell comprises an expression cassette comprising a promoter operably linked to a polynucleotide encoding the Cas9 polypeptide.

6. The method of claim 1, wherein the cell comprises the Cas9 polypeptide before the introducing of the Cas9-inhibiting polypeptide.

7. The method of claim 6, wherein the promoter is inducible and the method comprises contacting the cell with an agent or condition that induces expression of the Cas9 polypeptide in the cell prior to the introducing of the Cas9-inhibiting polypeptide.

8. The method of claim 1, wherein the cell comprises the Cas9 polypeptide after the introducing of the Cas9-inhibiting polypeptide.

9. The method of claim 8, wherein the promoter is inducible and the method comprises contacting the cell with an agent or condition that induces expression of the Cas9 polypeptide in the cell after the introducing of the Cas9-inhibiting polypeptide.

10. The method of claim 1, wherein the introducing of the Cas9-inhibiting polypeptide comprises expressing the Cas9-inhibiting polypeptide in the cell from an expression cassette that is present in the cell and is heterologous to the cell, wherein the expression cassette comprises a promoter operably linked to a polynucleotide encoding the Cas9-inhibiting polypeptide.

11. (canceled)

12. The method of claim 1, wherein the introducing of the Cas9-inhibiting polypeptide comprises introducing an RNA encoding the Cas9-inhibiting polypeptide into the cell and expressing the Cas9-inhibiting polypeptide in the cell from the RNA.

13. (canceled)

14. The method of claim 1, wherein the cell is a eukaryotic cell.

15-17. (canceled)

18. The method of claim 14, wherein the method occurs ex vivo.

19. The method of claim 18, wherein the cell is introduced into a mammal after the introducing of the Cas9-inhibiting polypeptide.

20-21. (canceled)

22. The method of claim 21, wherein the introducing of the Cas9-inhibiting polypeptide comprises introducing a polynucleotide encoding the Cas9-inhibiting polypeptide into a bacterial cell using bacteriophage, and expressing the Cas9-inhibiting polypeptide in the cell from the polynucleotide.

23. The method of claim 1, wherein the Cas9 polypeptide is SpyCas9, Efa1Cas9, or Efa3Cas9.

24. (canceled)

25. The cell of claim 1, wherein the cell is a eukaryotic cell.

26-28. (canceled)

29. A polynucleotide comprising a nucleic acid encoding a Cas9-inhibiting polypeptide, wherein the Cas9-inhibiting polypeptide is at least 95% identical to any one or more of SEQ ID NOS: 1-8.

30. The polynucleotide of claim 29 wherein the Cas9-inhibiting polypeptide inhibits a Cas9 polypeptide selected from the group consisting of SpyCas9, Efa1Cas9, and Efa3Cas9.

31-35. (canceled)

36. A vector comprising the polynucleotide of claim 29.

37-38. (canceled)

39. An isolated Cas9-inhibiting polypeptide, wherein the Cas9-inhibiting polypeptide is at least 95% identical to any one or more of SEQ ID NOS:1-8.

40-43. (canceled)
